(19) United States
(12) Patent Application Publication
(10) Pub. No.: US 2020/0241012 A1
(43) Pub. Date: Jul. 30, 2020

(54) RGMa Fragment Based Diagnostic Assay

(71) Applicant: ABBVIE DEUTSCHLAND GMBH & CO. KG, Wiesbaden (DE)

(72) Inventors: Stefan Barghorn, Ludwigshafen (DE); Bernhard Klaus Mueller, Ludwigshafen (DE); Martin Schmidt, Ludwigshafen (DE); Andreas Striebinger, Ludwigshafen (DE)

(21) Appl. No.: 16/560,632

(22) Filed: Sep. 4, 2019

Related U.S. Application Data
(63) Continuation of application No. 14/850,185, filed on Sep. 10, 2015, now abandoned.
(60) Provisional application No. 62/048,745, filed on Sep. 10, 2014.

(51) Int. Cl. G01N 33/68; G01N 33/50; A61K 31/58

(52) U.S. Cl. CPC G01N 33/6896; G01N 33/5023; G01N 2800/285; G01N 2800/52; G01N 2333/705; A61K 31/58

(57) ABSTRACT

Provided are diagnostic assays and method s of using the diagnostic assays for detecting and quantifying RGMa fragments in a sample. The methods may be used detection of the RGMa fragments to monitoring drug treatment and effectiveness of drug treatment in neurodegenerative diseases.

### CROSS-REFERENCE TO RELATED APPLICATION(S)

This claims priority to U.S. Provisional Patent Application No. 62/048,745, filed on Sep. 10, 2014, the entire contents of which are fully incorporated herein by reference.

### FIELD ON THE INVENTION

The present invention relates to an assay for detecting and determining RGMa fragments in a sample and the use of the assay in determining, optimizing, predicting, and monitoring a treatment in a subject suffering from a neurodegenerative disease.

### BACKGROUND

Multiple sclerosis (MS) is a chronic inflammatory disease of the central nervous system. Current available MRI techniques with gadolinium application visualize the various kinds of lesions occurring during the disease process and serve as a biological marker. However, investigations of cerebrospinal fluid (CSF) and proteins may provide further insight into the interplay between disease progression, chronic inflammation and response to treatment, such as intrathecal retarded release steroid applications.

The process of axonal regeneration improves clinical outcomes in multiple sclerosis. The presence of several inhibitors of regeneration is known in myelin and glial structures, i.e. myelin-associated glycoprotein; NogoA, OMgp (oligodendrocyte myelin glycoprotein). One further inhibitor is the repulsive guidance molecule A (RGMa), which also counteracts neuronal regeneration and functional recovery. RGMa is a glycosylphosphatidylinositol (GPI)-anchored glycoprotein that is a potent inhibitor of neurite outgrowth and emerges as an important factor inhibiting neuronal regeneration and functional recovery after CNS trauma or inflammation. RGMa exists in membrane-bound and soluble forms, which are both inhibitory for neurite growth. RGMa has been localized to CNS myelin, fresh lesions and mature scar tissue in humans suffering from traumatic brain injury or ischemic stroke.

RGMa and its fragments that are present in the brain and spinal cord may promote neurodegeneration and inhibit neuroregeneration. RGMa influences regeneration of nerve fibers and degeneration of neurons. ELISA-based assays have been used to detect RGMa species in order to identify inhibition of regeneration activity. However, these assays lack the ability to distinguish RGMa fragments of different sizes and usually do not show the level of high sensitivity required for detection of different RGMa fragments. In addition, ELISA-based assays are not able to detect RGMa because of the low amounts of protein in body fluids. A diagnostic assay is needed that has higher sensitivity and can detect and distinguish RGMa fragments as well as correlate the RGMa fragments with enhanced functional recovery and regeneration in patients suffering from MS or any other neurodegenerative disease.

### SUMMARY OF INVENTION

The present invention is directed to a method of detecting and quantifying at least one RGMa fragment in a sample. The method comprises (a) obtaining a sample from a subject comprising at least one RGMa fragment; (b) contacting the sample with a capture binding protein, wherein the capture binding protein binds to the at least one RGMa fragment to form a capture binding protein-RGMa fragment complex; (c) contacting the sample with a detection binding protein, wherein the detection binding protein interacts with the capture binding protein to form a detection binding protein-capture binding protein RGMa fragment complex, and (d) detecting and quantifying the at least one RGMa fragment in the sample. The at least one RGMa fragment may be a RGMa fragment having a size between about 1 kDa to about 65 kDa. The RGMa fragment may have a size of 10 kDa, 18 kDa, 20 kDa, 30 kDa, 40 kDa, 50 kDa, or 65 kDa. The RGMA fragment may be selected from the group consisting of 18 kDa RGMa fragment, 30 kDa RGMa fragment, and 40 kDa RGMa fragment. The at least one RGMa fragment may be separated using gel electrophoresis before step (b). At least two RGMa fragments may be detected. The at least two RGMa fragments may be 30 kDa and 40 kDa in size. At least three RGMa fragments may be detected. The at least three RGMa fragments may be 18 kDa, 30 kDa, and 40 kDa in size. The at least one RGMa fragment may be a soluble RGMa fragment. The size of the RGMa fragment may be determined by SDS-PAGE. The SDS PAGE may be 4-15%. The capture binding protein may be an RGMa-selective antibody. The antibody may be a biotinylated RGMa-selective antibody. The detection binding protein may be a tetravalent avidin and the detectable label may be a biotinylated horseradish peroxidase. The at least one RGMa fragment may be detected using a peroxidase staining kit. The RGMa fragment may be a human RGMa fragment. The method sample may comprise cerebrospinal fluid, blood, serum or plasma.

The present invention is directed to a method of detecting and quantifying at least one RGMa fragment in a sample. The method comprises (a) obtaining a sample from a subject comprising at least one RGMa fragment; (b) contacting the sample with a capture binding protein, wherein the capture binding protein binds to the at least one RGMa fragment to form a capture binding protein-RGMa fragment complex; (c) contacting the sample with a detection binding protein, wherein the detection binding protein interacts with the capture binding protein to form a detection binding protein-capture binding protein RGMa fragment complex, and (d) detecting and quantifying the at least one RGMa fragment in the sample. The at least one RGMa fragment may be a RGMa fragment having a size between about 1 kDa to about 65 kDa. The RGMa fragment may have a size of 10 kDa, 18 kDa, 20 kDa, 30 kDa, 40 kDa, 50 kDa, or 65 kDa. The RGMA fragment may be selected from the group consisting of 18 kDa RGMa fragment, 30 kDa RGMa fragment, and 40 kDa RGMa fragment. The at least one RGMa fragment may be separated using gel electrophoresis before step (b). The method further comprises immobilizing the at least one RGMa fragment to a membrane to generate a western blotting membrane before step (b); contacting the western blotting membrane with the capture binding protein, wherein the capture binding protein binds to the at least one RGMa fragment immobilized on the western blotting membrane to form a capture binding protein-RGMa fragment complex in step (b); and contacting the western blotting membrane with a detection binding protein, wherein the detection binding protein interacts with the capture binding protein to form a detection binding protein-capture binding protein RGMa fragment complex in step (c). At least two RGMa fragments may be detected. The at least two RGMa fragments may be 30 kDa and 40 kDa in size. At least three RGMa fragments may be detected. The at least three RGMa fragments may be 18 kDa, 30 kDa, and 40 kDa in size. The at least one RGMa fragment may be a soluble RGMa fragment. The size of the RGMa fragment may be determined by SDS-PAGE. The SDS PAGE may be 4-15%. The membrane may be a nitrocellulose membrane. The capture binding protein may be an RGMa-selective antibody. The antibody may be a biotinylated RGMa-selective antibody. The detection binding protein may be a tetravalent avidin and the detectable label may be a biotinylated horseradish peroxidase. The at least one RGMa fragment may be detected using a peroxidase staining kit. The RGMa fragment may be a human RGMa fragment. The method sample may comprise cerebrospinal fluid, blood, serum or plasma.

The present invention is directed to a method of detecting and quantifying at least one RGMa fragment in a sample. The method comprises (a) obtaining a sample from a subject comprising at least one RGMa fragment; (b) contacting the sample with a capture binding protein, wherein the capture binding protein binds to the at least one RGMa fragment to form a capture binding protein-RGMa fragment complex; (c) contacting the sample with a detection binding protein, wherein the detection binding protein interacts with the capture binding protein to form a detection binding protein-capture binding protein RGMa fragment complex, and (d) detecting and quantifying the at least one RGMa fragment in the sample. The at least one RGMa fragment may be a RGMa fragment having a size between about 1 kDa to about 65 kDa. The RGMa fragment may have a size of 10 kDa, 18 kDa, 20 kDa, 30 kDa, 40 kDa, 50 kDa, or 65 kDa. The RGMA fragment may be selected from the group consisting of 18 kDa RGMa fragment, 30 kDa RGMa fragment, and 40 kDa RGMa fragment. The at least one RGMa fragment may be separated using gel electrophoresis before step (b). The method further comprises immobilizing the at least one RGMa fragment to a membrane to generate a western blotting membrane before step (b); contacting the western blotting membrane with the capture binding protein, wherein the capture binding protein binds to the at least one RGMa fragment immobilized on the western blotting membrane to form a capture binding protein-RGMa fragment complex in step (b); and contacting the western blotting membrane with a detection binding protein, wherein the detection binding protein interacts with the capture binding protein to form a detection binding protein-capture binding protein RGMa fragment complex in step (c). At least two RGMa fragments may be detected. The at least two RGMa fragments may be 30 kDa and 40 kDa in size. At least three RGMa fragments may be detected. The at least three RGMa fragments may be 18 kDa, 30 kDa, and 40 kDa in size. The at least one RGMa fragment may be a soluble RGMa fragment. The method further comprises separating a RGMa protein standard on the gel concurrently with the proteins in the sample in step (b); and (g) comparing the at least one RGMa fragment with the separated RGMa protein standard to quantify the fragments. The RGMa protein standard may be a gradient of recombinant RGMa fragments. The gradient may comprise the RGMa protein standard 10, 25, 50, 100, and 200 pg/mL. The size of the RGMa fragment may be determined by SDS-PAGE. The SDS PAGE may be 4-15%. The membrane may be a nitrocellulose membrane. The capture binding protein may be an RGMa-selective antibody. The antibody may be a biotinylated RGMa-selective antibody. The detection binding protein may be a tetravalent avidin and the detectable label may be a biotinylated horseradish peroxidase. The at least one RGMa fragment may be detected using a peroxidase staining kit. The RGMa fragment may be a human RGMa fragment. The method sample may comprise cerebrospinal fluid, blood, serum or plasma.

The present invention is directed to a method of determining the effectiveness of a treatment for a neurodegenerative disease in a subject in need thereof. The method comprises (a) determining the level of at least one RGMa fragment in a sample from the subject using the method of any one of claims 1 to 21; and (b) comparing the level of the at least one RGMa fragment in a sample from the subject to a control level of the at least one RGMa fragment, wherein if the level of the at least one fragment is increased compared to the control level, the treatment is determined to be ineffective in treating the neurodegenerative disease, and wherein if the level of the at least one fragment is the same or decreased compared to the control level, the treatment is determined to be effective in treating the neurodegenerative disease. The control level of the at least one RGMa fragment may be the level of the at least one RGMa fragment in a subject that has the neurodegenerative disease but has not been treated with for the neurodegenerative disease. The treatment may comprise a, neurorestorative drug, neuroprotective drug, or neuroregenerative drug. The treatment may comprise at least one of triamcinolone acetonide (TCA), Tecfidera/BG-12 (dimethyl fumarate), Gilenya (fingolimod), Laquinimod, B-Interferons, Copaxone, Daclizumab, Alemtuzumab, Rituximab, or combinations thereof. The treatment may comprise triamcinolone acetonide (TCA). At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of determining the effectiveness of a treatment for a neurodegenerative disease in a subject in need thereof. The method comprises (a) determining the level of at least one RGMa fragment in a sample from the subject using the method of any one of claims 1 to 21; and (b) comparing the level of the at least one RGMa fragment in a sample from the subject to a control level of the at least one RGMa fragment, wherein if the level of the at least one fragment is increased compared to the control level, the treatment is determined to be ineffective in treating the neurodegenerative disease, and wherein if the level of the at least one fragment is the same or decreased compared to the control level, the treatment is determined to be effective in treating the neurodegenerative disease. The method further comprises continuing to administer the treatment determined to be effective in treating the neurodegenerative disease to the subject in need thereof. The control level of the at least one RGMa fragment may be the level of the at least one RGMa fragment in a subject that has the neurodegenerative disease but has not been treated with for the neurodegenerative disease. The treatment may comprise a, neurorestorative drug, neuroprotective drug, or neuroregenerative drug. The treatment may comprise at least one of triamcinolone acetonide (TCA), Tecfidera/BG-12 (dimethyl fumarate), Gilenya (fingolimod), Laquinimod, B-Interferons, Copaxone, Daclizumab, Alemtuzumab, Rituximab, or combinations thereof. The treatment may comprise triamcinolone acetonide (TCA). At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of predicting the responsiveness of a subject suffering from a neurodegenerative disease to a treatment. The method comprises (a) determining the levels of at least one RGMa fragment in a sample from the subject using the method of any one of claims 1 to 21; (b) comparing the levels of the at least one RGMa fragment in a sample from the subject to a control level of the at least one RGMa fragment; and (c) providing a prediction of responsiveness of the subject to a treatment if the levels of the at least one RGMa fragment in a sample are decreased compared to the control levels. The treatment may comprise a, neurorestorative drug, neuroprotective drug, or neuroregenerative drug. The treatment may comprise at least one of triamcinolone acetonide (TCA), Tecfidera/BG-12 (dimethyl fumarate), Gilenya (fingolimod), Laquinimod, β-Interferons, Copaxone, Daclizumab, Alemtuzumab, Rituximab, or combinations thereof. The treatment may comprise triamcinolone acetonide (TCA). At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of predicting the responsiveness of a subject suffering from a neurodegenerative disease to a treatment. The method comprises (a) determining the levels of at least one RGMa fragment in a sample from the subject using the method of any one of claims 1 to 21; (b) comparing the levels of the at least one RGMa fragment in a sample from the subject to a control level of the at least one RGMa fragment; and (c) providing a prediction of responsiveness of the subject to a treatment if the levels of the at least one RGMa fragment in a sample are decreased compared to the control levels. The method further comprises administering the treatment to the subject predicted to be responsive to the treatment. The treatment may comprise a, neurorestorative drug, neuroprotective drug, or neuroregenerative drug. The treatment may comprise at least one of triamcinolone acetonide (TCA), Tecfidera/BG-12 (dimethyl fumarate), Gilenya (fingolimod), Laquinimod, β-Interferons, Copaxone, Daclizumab, Alemtuzumab, Rituximab, or combinations thereof. The treatment may comprise triamcinolone acetonide (TCA). At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of treating a subject suffering from neurodegenerative disease. The method comprises (a) determining the levels of at least one RGMa fragment in a sample from the subject using the method of any one of claims 1 to 21, (b) comparing the levels of the at least one RGMa fragment in a sample from the subject to a control level of the at least one RGMa fragment; and (c) administering a treatment regimen to the subject if the levels of the fragments are increased compared to control levels. The treatment may comprise a, neurorestorative drug, neuroprotective drug, or neuroregenerative drug. The treatment may comprise at least one of triamcinolone acetonide (TCA), Tecfidera/BG-12 (dimethyl fumarate), Gilenya (fingolimod), Laquinimod, β-Interferons, Copaxone, Daclizumab, Alemtuzumab, Rituximab, or combinations thereof. The treatment may comprise triamcinolone acetonide (TCA). At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of optimizing a treatment regimen for a subject suffering from a neurodegenerative disease. The method comprises (a) determining a first level of at least one RGMa fragment in a first sample from the subject using the method of any one of claims 1 to 20, wherein the first sample is taken from the subject at a time point before or during the period when the subject has begun a treatment regimen; (b) determining a second level of the at least one RGMa fragment in second sample from the subject at a time later than step (a), wherein an decrease in the second level of the at least one RGMa fragment compared to the first level of the at least one RGMa fragment indicates the treatment regimen has a therapeutic efficacy against the neurodegenerative disease; (c) determining the levels of at least one RGMa fragment in a first sample from the subject using the method of claim 1, (d) comparing the levels of the at least one RGMa fragment in a sample from the subject to a control level of the at least one RGMa fragment; and (e) providing a prediction of responsiveness of the subject to a treatment if the levels of the at least one RGMa fragment in a sample are decreased compared to the control levels. The treatment regimen may be a neurorestorative treatment regimen. The success rate of the neurorestorative treatment regimen may be increased. The treatment regimen may be a neuroprotective treatment regimen. The success rate of the neuroprotective treatment regimen may be increased. At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of monitoring a regeneration-promoting drug treatment of a subject suffering from neurodegenerative disease. The method comprises (a) determining a first level of at least one RGMa fragment in a first sample from the subject using the method of any one of claims 1 to 21, wherein the first sample is taken from the subject at a time point before or during the period when the subject has begun drug treatment; (b) determining a second level of the at least one RGMa fragment in second sample from the subject at a time later than step (a), wherein an decrease in the second level of the at least one RGMa fragment compared to the first level of the at least one RGMa fragment indicates the drug treatment regimen has a therapeutic efficacy against the neurodegenerative disease, and an increase in the second level of the at least one RGMa fragment compared to the first level of the at least one RGMa fragment indicates the drug treatment regimen does not have a therapeutic efficacy against the neurodegenerative disease; and (c) administering a different drug treatment to the subject if the drug treatment regimen does not have a therapeutic efficacy against the neurodegenerative disease. At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

The present invention is directed to a method of screening a compound for therapeutic efficacy against a neurodegenerative disease. The method comprises (a) determining a first level of at least one RGMa fragment in a sample comprising cells using the method of any one of claims 1 to 21; (b) contacting the sample with a compound, (c) determining a second level of at least one RGMa fragment in second sample from the subject at a time later than step (b), wherein an decrease in the second level of the at least one RGMa fragment compared to the first level of the at least one RGMa fragment indicates the compound as having therapeutic efficacy against the neurodegenerative disease, and wherein an increase in the second level of the at least one RGMa fragment compared to the first level of the at least one RGMa fragment indicates the compound as not having therapeutic efficacy against the neurodegenerative disease; and (d) selecting the compound identified as having therapeutic efficacy. At least two RGMa fragment may be detected. The at least two RGMa fragments may be 30 kDa and 40 kDa in size. At least three RGMa fragments may be detected. The at least three RGMa fragments may be 18 kDa, 30 kDa, and 40 kDa in size. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy. The neurodegenerative disease or disorder may be multiple sclerosis. The RGMa fragment may be a human RGMa fragment. The method sample may comprise cerebrospinal fluid, blood, serum or plasma.

### BRIEF DESCRIPTION OF THE DRAWINGS

FIG. 1 shows cleavage of RGMa by the proprotein convertases SKI-1 and Furin generating fragments of 18, 30, and 40 kDa (a, b, c, solid arrows).

FIG. 2 shows RGMa fragments present in CSF of progressive MS patients.

FIG. 3 A, B, C shows clinical data of 17 patients with an immediate response during TCA therapy with improved EDSS (FIG. 3A), walking distance (FIG. 3B) and walking speed (FIG. 3C). All data are given as mean±SEM (standard error of means); *=p<0.05; **=p<0.01; ***=p<0.001; *=significance level of p-values from the post hoc analysis; I=baseline before the first TCA administration, II, before the second TCA administration, III=before the third TCA administration, IV=before the fourth TCA administration, V=before the fifth TCA application; VI=before the sixth TCA application.

FIG. 4 A, B, C shows clinical data on the 8 patients without immediate response to repeat TCA application with no improved EDSS (FIG. 4A), walking distance (FIG. 4B) and walking speed (FIG. 4C). All data are given as mean±SEM (standard error of means); I=baseline before the first TCA administration, II=before the second TCA administration, III=before the third TCA administration, IV=before the fourth TCA application; V=before the fifth TCA application.

FIG. 5 A, B, C show the change of the RGMa concentrations (40 kDa (FIG. 5A); 30 kDa (FIG. 5B) and protein concentrations (FIG. 5C) in cerebrospinal fluid of immediate responders to TCA therapy. All data are given as mean±SEM; *=p<0.05; **=p<0.01; ***=p<0.001; *=significance level of p-values from the post hoc analysis; I=baseline before the first TCA administration; II, before the second TCA administration; III, before the third TCA administration; IV, before the fourth TCA administration.

FIG. 6 shows three representative Western blots with histograms for the densitometric analysis of RGMa CSF levels taken from the 17 patients with an immediate response to TCA application.

FIG. 7 A, B, C show RGMa concentrations (40 kDa (FIG. 7A); 30 kDa (FIG. 7B) and protein concentrations (FIG. 7C) in cerebrospinal fluid of not immediate responding MS patients to TCA therapy. All data are given as mean±SEM; *=p<0.05; **=p<0.01; ***=p<0.001; *=significance level of p-values from the post hoc analysis; I=baseline before the first TCA administration; II=before the second TCA administration; III=before the third TCA administration; IV=before the fourth TCA administration.

FIG. 8 shows three representative Western blots with histograms for the densitometric analysis of RGMa CSF levels taken from the 8 patients without a prompt response to TCA application.

### DETAILED DESCRIPTION

The present invention is directed to an assay for analyzing the levels of RGMa fragments, and determining, optimizing, predicting, and monitoring a treatment regimen for a neurodegenerative disease in a subject in need thereof. The RGMa fragment based diagnostic assay can be used to detect specific RGMa fragments of a particular size. The immunodetection of endogenous and recombinant RGMa fragments may be used to determine, optimize, predict, and monitor a treatment in a subject suffering from or showing symptoms of a neurodegenerative disease. The RGMa fragment based diagnostic assay quantitatively measures the concentration of soluble regeneration-inhibitory RGMa fragments present in human bodily fluids like CSF, blood, serum, and plasma using minimal amounts. This diagnostic assay provides a higher sensitivity (detection of low picogram (pg) amounts of RGMa in human material) and is, in combination with the RGMa protein standard, a quantitative tool to identify the RGMa concentration in body fluids of patients suffering from neurodegenerative diseases such as multiple sclerosis. Additionally, this assay distinguishes different fragments of RGMa, and allows for monitoring of pattern shifts of these fragments during disease progression. Therefore, this method is superior over the current technologies investigating only the total RGM protein as it provides a means for patient stratification in neurorestorative drug trials; a means to follow patients which may respond positively to regeneration-promoting drugs; a means to identify non-responders in such trials; a means to optimize neurorestorative treatment strategies; and a means to increase success rate of neurorestorative drug approaches.

Section headings as used in this section and the entire disclosure herein are merely for organization purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the terms “including” and “having,” as well as other forms of those terms, such as “includes,” “included”, “has,” and “have” are not limiting.

As used throughout the specification and the claims, the following terms have the following meanings:

The term “control subject” as used herein means a healthy subject, i.e. a subject having no clinical signs or symptoms of a neurodegenerative disease, such as multiple sclerosis (MS). The control subject is clinically evaluated for otherwise undetected signs or symptoms of MS, which evaluation may include routine physical examination and/or laboratory testing. A “control group” as used herein refers to a group of control subjects or healthy subjects, i.e. a group of subjects who have no clinical signs or symptoms of the neurodegenerative disease, such as MS.

“Sample,” “biological sample,” “test sample,” “specimen,” “sample from a subject,” and “patient sample” as used herein may be used interchangeable and may be a sample of blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “subject”, “patient” or “subject in the method” as used herein interchangeably, means any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human. In some embodiments, the subject or subject may be a human or a non-human. In some embodiments, the subject may be a human subject at risk or suspected at being at risk for developing or already having a neurodegenerative disease, such as MS.

The terms “treat,” “treated,” or “treating” as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. RGMa Fragment-Based Diagnostic Assay

The present invention is directed to diagnostic assays for quantifying and detecting RGMa fragments in a sample. The RGMa may be any RGMa fragment. The diagnostic assay may quantify and detect at least one RGMa fragment. RGMa is synthesized as a 450 amino acid (aa) preproprotein that contains a 47 aa signal sequence, a 121 aa N-terminal prosegment, a 256 mature region, and a 26 aa C-terminal prosegment. The N-terminal prosegment contains an RGD tripeptide and the molecule's only two potential N-linked glycosylation sites. The mature segment shows an abbreviated shortened domain with structural homology to the von Willebrand factor domain. Proteolytic processing occurs at an aspartic acid-proline bond, creating a predicted 32 kDa mature region. The GPI-anchored RGMa protein is processed by Furin and the proprotein convertase SKI-1 into numerous membrane-bound and soluble fragments and this processing is required for their proper in vivo functions.

The receptor for RGMa is reported to be neogenin. RGM-A has also been shown to be a bone morphogenic protein coreceptor, able to bind both BMP-2, BMP-4, BMP-5, and BMP-6. Several different fragments of RGMa exert their neurite growth inhibitory function by binding to their neuronal receptor Neogenin. Neogenin is a member of the immunoglobulin superfamily and consists of four N-terminal immunoglobulin-like domains (Ig), six fibronectin type III (FNIII) domains, a transmembrane domain and a C-terminal internal domain. Two different RGMa fragments, the N-terminal (30 kDa) and the C-terminal fragment (40 kDa) bind to the same FNIII domain (domain 3-4) of Neogenin, despite their lack of sequence homology. RGMa fragments have been shown to inhibit neurite growth in vitro. Neutralization of RGMa activity with a polyclonal RGMa antibody in a spinal cord injury model resulted in long distance axon regeneration and improved functional recovery. In cerebral stroke models down regulation of RGMa resulted in neuroprotection and enhanced functional recovery acting via Neogenin which is well known for its fundamental role in axon guidance and cellular differentiation. The presence of the two regeneration inhibitory RGMa fragments (30 and 40 kDa) in human CSF suggests that these proteins contribute to regeneration failure and neurodegeneration in progressive MS patients. In MS patients, RGMa is expressed by immature and mature dendritic cells in brain and the spinal cord. RGMa may also have a role in the immune system, e.g. also on microglia cells or in the modulation of T cell responses as it is expressed on CD68-positive macrophages and on CD4-positive T-lymphocytes. In the brain, activated microglia cells express RGMa on their surface and decrease of microglial RGMa expression results in enhanced axonal growth both in vitro and in vivo. In addition, the RGMa gene was identified as a disease-associated gene in MS patients and certain rat strains induced with experimental autoimmune encephalomyelitis.

The diagnostic assay includes obtaining a sample from a subject comprising at least one RGMa fragment; contacting the sample with a capture binding protein, wherein the capture binding protein binds to the at least one RGMa fragment to form a capture binding protein-RGMa fragment complex; contacting the sample with a detection binding protein, wherein the detection binding protein interacts with the capture binding protein to form a detection binding protein-capture binding protein RGMa fragment complex, and detecting and quantifying the at least one RGMa fragment in the sample. The at least one RGMa fragment may have a size between about 1 kDa to about 65 kDa. The at least one RGMa fragment may have a size of about 10 kDa, about 18 kDa, about 20 kDa, about 30 kDa, about 40 kDa, about 50 kDa, or about 65 kDa. The at least one RMGa fragment may be selected from the group consisting of 18 kDa RGMa fragment, 30 kDa RGMa fragment, and 40 kDa RGMa fragment. The at least one RGMa fragment may be separated from other components of the sample, such as other RGMa fragments of different sizes. In some embodiments, the assay involves separating the fragments by size using a separation technique such as gel electrophoresis, column chromatography, and mass spectrometry.

a. RGMa Fragments

RGMa is detected by the diagnostic assay. The RGMa may be a RGMa fragment. The assay may detect at least one RGMa fragment.

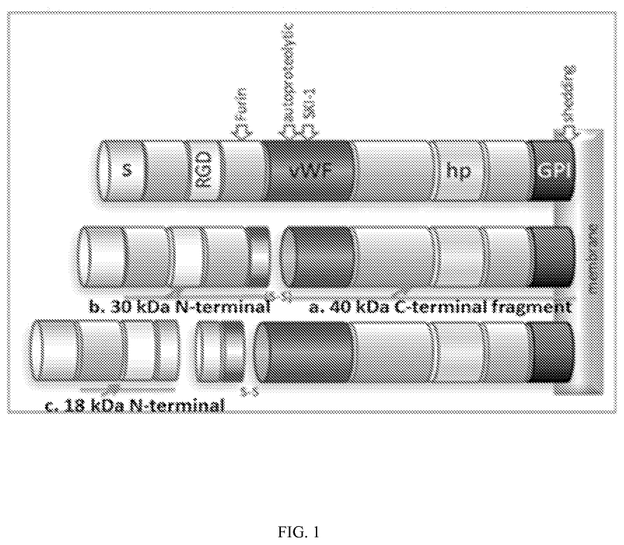

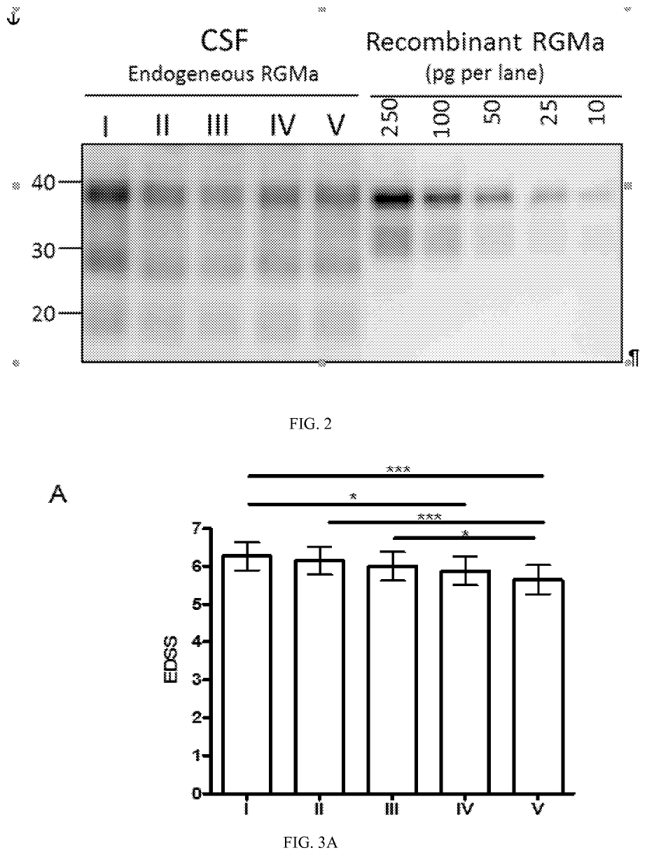

RGMa is cleaved at N-terminal amino acid 168 and within the N-terminal domain by two proteases, proprotein convertase SKI-1 and Furin, to yield the functionally active protein and active fragments of 18, 30 and 40 kDa (FIG. 1). The 30 kDa fragment is linked to the membrane-bound C-terminal 40 kDa fragment via disulfide bonds (S—S). Cleavage within the C-terminal (arrow, shedding) GPI-anchor domain results in release of the three fragments creating soluble forms of RGMa. Soluble forms of these fragments are generated when cleavage occurs in the C-terminus by sheddases and enzymes cleaving the GPI-anchor. Like the membrane-anchored form, all three soluble fragments (18 kDa=truncated N-terminal domain, 30 kDa=N-terminal domain, 40 kDa=C-terminal domain) are active as neurite growth and regeneration inhibitors.

The RGMa fragment based diagnostic assay may detect at least one RGMa fragment having a size between about 1 kDa and about 65 kDa. The RGMa fragment may be about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 4 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, about 60 kDa, about 61 kDa, about 62 kDa, about 63 kDa, about 64 kDa, about 65 kDa, or combinations thereof.

The RGMa fragment based diagnostic assay may detect at least one RGMa fragment, at least two RGMa fragments, at least three RGMa fragments, at least four RGMa fragments, at least five RGMa fragment, at least six RGMa fragments, or at least seven RGMa fragments. The RGMa fragment based diagnostic assay may detect 10 kDa RGMa fragment, 18 kDa RGMa fragment, 20 kDa RGMa fragment, 30 kDa RGMa fragment, 40 kDa RGMa fragment, 50 kDa RGMa fragment, 65 kDa RGMa fragment, or a combination thereof. For example, the RGMa fragment based diagnostic assay may detect 10 kDa RGMa fragment; 18 kDa RGMa fragment; 20 kDa RGMa fragment, 30 kDa RGMa fragment; 40 kDa RGMa fragment; 50 kDa RGMa fragment; 65 kDa RGMa fragment; 10 kDa RMGa fragment and 18 kDa RGMa fragment; 10 kDa RMGa fragment and 20 kDa RGMa fragment; 10 kDa RMGa fragment and 30 kDa RGMa fragment; 10 kDa RMGa fragment and 40 kDa RGMa fragment; 10 kDa RMGa fragment and 50 kDa RGMa fragment; 10 kDa RMGa fragment and 60 kDa RGMa fragment; 18 kDa RMGa fragment and 20 kDa RGMa fragment; 18 kDa RMGa fragment and 30 kDa RGMa fragment; 18 kDa RMGa fragment and 40 kDa RGMa fragment; 18 kDa RMGa fragment and 50 kDa RGMa fragment; 18 kDa RMGa fragment and 60 kDa RGMa fragment; 20 kDa RMGa fragment and 30 kDa RGMa fragment; 20 kDa RMGa fragment and 40 kDa RGMa fragment; 20 kDa RMGa fragment and 50 kDa RGMa fragment; 20 kDa RMGa fragment and 60 kDa RGMa fragment; 30 kDa RMGa fragment and 40 kDa RGMa fragment; 30 kDa RMGa fragment and 50 kDa RGMa fragment; 30 kDa RMGa fragment and 60 kDa RGMa fragment; 40 kDa RMGa fragment and 50 kDa RGMa fragment; 40 kDa RMGa fragment and 60 kDa RGMa fragment; 50 kDa RMGa fragment and 60 kDa RGMa fragment; 10 kDa RGMa fragment and at least two, at least three, at least four, at least five, or at least six of 18 kDa RGMa fragment, 20 kDa RGMa fragment, 30 kDa RGMa fragment, 40 kDa RGMa fragment, 50 kDa RGMa fragment, or 65 kDa RGMa fragment; 18 kDa RGMa fragment and at least two, at least three, at least four, at least five, or at least six of 10 kDa RGMa fragment, 20 kDa RGMa fragment, 30 kDa RGMa fragment, 40 kDa RGMa fragment, 50 kDa RGMa fragment, or 65 kDa RGMa fragment; 20 kDa RGMa fragment and at least two, at least three, at least four, at least five, or at least six of 10 kDa RGMa fragment, 18 kDa RGMa fragment, the 30 kDa RGMa fragment, 40 kDa RGMa fragment, 50 kDa RGMa fragment, or 65 kDa RGMa fragment; 30 kDa RGMa fragment and at least two, at least three, at least four, at least five, or at least six of 10 kDa RGMa fragment, 18 kDa RGMa fragment, 20 kDa RGMa fragment, 40 kDa RGMa fragment, 50 kDa RGMa fragment, or 65 kDa RGMa fragment; 40 kDa RGMa fragment and at least two, at least three, at least four, at least five, or at least six of 10 kDa RGMa fragment, 18 kDa RGMa fragment, 20 kDa RGMa fragment, 30 kDa RGMa fragment, 50 kDa RGMa fragment, or 65 kDa RGMa fragment; 50 kDa RGMa fragment and at least two, at least three, at least four, at least five, or at least six of 10 kDa RGMa fragment, 18 kDa RGMa fragment, 20 kDa RGMa fragment, 30 kDa RGMa fragment, 40 kDa RGMa fragment, or 65 kDa RGMa fragment; 65 kDa RGMa fragment and at least two, at least three, at least four, at least five, or at least six of 10 kDa RGMa fragment, 18 kDa RGMa fragment, 20 kDa RGMa fragment, 30 kDa RGMa fragment, 40 kDa RGMa fragment, or 50 kDa RGMa fragment. The RGMa fragment based diagnostic assay may detect 18 kDa RGMa fragment, 30 kDa RGMa fragment, and 40 kDa RGMa fragment as long as these fragments retain the binding epitope sites for the capture binding proteins such as the anti-RGMa-antibody as discussed below.

b. Fragment Detection

The RGMa fragments may be detected and quantified in a sample from a subject by various means to separate the fragments and determine the size of the fragment(s). The fragments may be detected using a capture binding protein, such as an RGMa fragment binding protein, such as an anti-RGMa antibody, that bind specifically to the RGMa fragment. The capture binding protein may have a detectable label or is recognized by a detection binding protein that has a detectable label. The detectable label allows the identification of the RGMa fragment.

In some embodiments, the RGMa fragments are identified, sized and quantified using SDS-PAGE/Western blotting analysis. In some embodiments, the RGMa fragments are identified, sized and quantified using a column chromatography technique. In some embodiments, the RGMa fragments are identified, sized and quantified using mass spectrometry.

The capture binding protein may be an anti-RGMa antibody, such as a biotinylated RGMa-selective antibody (BAF2459 R&D Systems) or RGMa antibodies described in U.S. Patent Publication Nos. 2004/0102376, 2010/0028340, 2011/0135664, 2013/0330347, and 2014/0023659. Antibody-binding to the RGMa fragment may be visualized after incubation with the ABC Peroxidase Staining Kit (Pierce; 32020) or high sensitive ECL solution (Thermo Scientific, SuperSignal West Femto Chemiluminescence Substrate, 34094) and scanned with VersaDoc Imager (BioRad). Quantity One Version 4.6.9 (BioRad) may be used to quantify band intensities of recombinant RGMa (R&D Systems, 2459-RM-050) and the single RGMa fragments in the body fluids.

(1) SDS-PAGE/Western Blotting

The RGMa fragment based diagnostic assay may further include immobilizing the at least one RGMa fragment to a membrane to generate a western blotting membrane, contacting the western blotting membrane with the capture binding protein, wherein the capture binding protein binds to the at least one RGMa fragment immobilized on the western blotting membrane to form a capture binding protein-RGMa fragment complex; and contacting the western blotting membrane with a detection binding protein, wherein the detection binding protein interacts with the capture binding protein to form a detection binding protein-capture binding protein RGMa fragment complex.

An RGMa protein standard marker may be used and separated on the SDS-PAGE at the same time as the sample. The at least one RGMa fragment band intensity is compared to the RGMa protein standard marker to determine the size of the RGMa fragment and/or quantify the amount of the at least one RGMa fragment. The RGMa protein standard may be a gradient of recombinant RGMa fragments. In some embodiments, the gradient of recombinant RGMa fragments includes 10, 25, 50, 100, and 200 pg/mL. The SDS-PAGE may have between 5% to 25% acrylamide. In some embodiments, the SDS-PAGE may be a 4-15% acrylamide gradient gel. The membrane may be nitrocellulose or PVDF membrane.

### 3. Methods of Using the RGMa Fragment Based Diagnostic Assay—Methods of Diagnosing, Prognosticating, or Assessing the Efficacy of a Therapeutic/Prophylactic Treatment

Also provided herein is a method of using the RGMa fragment based diagnostic assay. The method includes obtaining a sample from the subject in need thereof. The method utilizes the RGMa fragment based diagnostic assay to detect the presence and/or level of at least one of the above-described RGMa fragments in the sample obtained from the subject. The subject may be suffering or at risk of suffering from a neurodegenerative disease.

The method utilizes the RGMa fragment based diagnostic assay to determine the effectiveness of a treatment or treatment regimen for the neurodegenerative disease. In other embodiments, the method utilizes the RGMa fragment based diagnostic assay to predict the responsiveness of the subject suffering from the neurodegenerative disease to the treatment or treatment regimen. In some embodiments, the method utilizes the RGMa fragment based diagnostic assay to determine if the treatment or treatment regimen should be administered to the subject. In still other embodiments, the method utilizes the RGMa fragment based diagnostic assay to optimize the treatment or treatment regimen for the subject suffering from the neurodegenerative disease. In some embodiments, the method may use the RGMa fragment based diagnostic assay to monitoring the treatment or treatment regimen of the subject suffering from the neurodegenerative disease. In other embodiments, the method utilizes the RGMa fragment based diagnostic assay to screen for a compound that is therapeutically effective against the neurodegenerative disease.

a. Neurodegenerative Diseases

RGMa may play a role as a modulator of the interplay between neurodegeneration and progression of chronic disease on the one hand and regeneration on the other hand. The neurodegenerative disease may be a disease in which the presence of RGMa is associated with the disease, i.e., wherein RGMa activity is detrimental. For example, RGMa has been found in ischemia damaged human brain tissue, in the lesions of humans suffering from traumatic brain injury, in the plaque regions of AD patients, in the substantia nigra of Parkinson's disease patients and in MS patients. The neurodegenerative disease or disorder may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, traumatic injury to the CNS, such as spinal cord injury, traumatic brain injury, and stroke, such as an ischemic cerebral stroke, glaucoma, diabetic retinopathy, age-dependent macular degeneration, and a leukodystrophy.

(1) Multiple Sclerosis

Multiple sclerosis (MS) is a disabling disease of the central nervous system that disrupts the flow of information within the brain, and between the brain and body. MS involves an immune-mediated process in which an abnormal response of the body's immune system is directed against the central nervous system (CNS), which is made up of the brain, spinal cord, retina, and optic nerves. This disorder is characterized by various subtypes regarding its progression and its predominant cerebral and spinal localization of inflammatory lesions. Such subtypes include relapsing-remitting MS (RRMS), secondary-progressive MS (SPMS), primary-progressive MS (PPMS), and progressive-relapsing MS (PRMS).

RRMS is characterized by clearly defined attacks of worsening neurologic function, often called relapses, flare-ups or exacerbations, which are followed by partial or complete recovery periods (remissions), during which symptoms improve partially or completely, and there is no apparent progression of disease. SPMS is characterized as the second phase of MS and occurs in individuals who initially had a RRMS disease course. Individuals with SPMS may or may not continue to experience relapses caused by inflammation as the disease gradually changes from the inflammatory process seen in RRMS to a more steadly progressive phase characterized by nerve damage or loss. PPMS is characterized by steady worsening of neurologic functioning, without any distinct relapses or periods of remission. An individual with PPMS has a rate of progression that may vary over time with occasional plateaus or temporary improvement but the progression is continuous. PRMS is similar to PPMS in that individuals with PRMS experience steadily worsening neurologic function from the very beginning, in addition to occasional relapses like those experienced by people with RRMS.

The term “clinically isolated syndrome” (CIS) is used to describe a first episode of neurologic symptoms that lasts at least 24 hours and is caused by inflammation and demyelination in one or more sites in the central nervous system. CIS can be either monofocal, where the person experiences a single neurologic sign or symptom that is caused by a single lesion, or multifocal, where the person experiences more than one sign or symptom, caused by lesions in more than one place. Persons who experience a CIS may or may not go on to develop MS. In the long run, most patients end up in a progressive, smoldering, chronic inflammatory process with neurodegenerative properties.

Subjects suffering from or suspected of suffering from MS may be assessed or diagnosed using Expanded Disability Status Score (EDSS) and/or an assessment of maximum walking distance and/or walking speed. In some embodiments, subjects having a decreased EDSS score, increased walking distance, and/or decreased walking speed may indicate that the treatment or treatment regimen is effective to the subject.

For example, an EDSS score of a subject that decreases at least about 0.1, at least about 0.2, at least about 0.3, at least about 0.4, at least about 0.5, at least about 0.6, at least about 0.7, at least about 0.8, at least about 0.9, at least about 1.0, at least about 2.0, at least about 3.0, at least about 4.0, at least about 5.0, or at least about 6.0 compared to the EDSS score of the subject before treatment indicates the treatment or treatment regimen is effective in treating the neurodegenerative disease.

For example, a subject having an increase in walking distance of between about 70 m to about 150 m, about 70 m to about 145 m, about 70 m to about 140 m, about 70 m to about 135 m, about 70 m to about 130 m, about 70 m to about 125 m, about 70 m to about 120 m, about 70 m to about 115 m, about 70 m to about 110 m, about 70 m to about 105 m, about 70 m to about 100 m, about 70 m to about 95 m, about 70 m to about 90 m, about 70 m to about 85 m, about 70 m to about 80 m, about 70 m to about 75 m, about 75 m to about 150 m, about 75 m to about 145 m, about 75 m to about 140 m, about 75 m to about 135 m, about 75 m to about 130 m, about 75 m to about 125 m, about 75 m to about 120 m, about 75 m to about 115 m, about 75 m to about 110 m, about 75 m to about 105 m, about 75 m to about 100 m, about 75 m to about 95 m, about 75 m to about 90 m, about 75 m to about 85 m, about 75 m to about 80 m, about 80 m to about 150 m, about 80 m to about 145 m, about 80 m to about 140 m, about 80 m to about 135 m, about 80 m to about 130 m, about 80 m to about 125 m, about 80 m to about 120 m, about 80 m to about 115 m, about 80 m to about 110 m, about 80 m to about 105 m, about 80 m to about 100 m, about 80 m to about 95 m, about 80 m to about 90 m, about 80 m to about 85 m, about 85 m to about 150 m, about 85 m to about 145 m, about 85 m to about 140 m, about 85 m to about 135 m, about 85 m to about 130 m, about 85 m to about 125 m, about 85 m to about 120 m, about 85 m to about 115 m, about 85 m to about 110 m, about 85 m to about 105 m, about 85 m to about 100 m, about 85 m to about 95 m, about 85 m to about 90 m, about 90 m to about 150 m, about 90 m to about 145 m, about 90 m to about 140 m, about 90 m to about 135 m, about 90 m to about 130 m, about 90 m to about 125 m, about 90 m to about 120 m, about 90 m to about 115 m, about 90 m to about 110 m, about 90 m to about 105 m, about 90 m to about 100 m, about 90 m to about 95 m, about 95 m to about 150 m, about 95 m to about 145 m, about 95 m to about 140 m, about 95 m to about 135 m, about 95 m to about 130 m, about 95 m to about 125 m, about 95 m to about 120 m, about 95 m to about 115 m, about 95 m to about 110 m, about 95 m to about 105 m, about 95 m to about 100 m, about 100 m to about 150 m, about 100 m to about 145 m, about 100 m to about 140 m, about 100 m to about 135 m, about 100 m to about 130 m, about 100 m to about 125 m, about 100 m to about 120 m, about 100 m to about 115 m, about 100 m to about 110 m, about 100 m to about 105 m, about 105 m to about 150 m, about 105 m to about 145 m, about 105 m to about 140 m, about 105 m to about 135 m, about 105 m to about 130 m, about 105 m to about 125 m, about 105 m to about 120 m, about 105 m to about 115 m, about 105 m to about 110 m, about 110 m to about 150 m, about 110 m to about 145 m, about 110 m to about 140 m, about 110 m to about 135 m, about 110 m to about 130 m, about 110 m to about 125 m, about 110 m to about 120 m, about 110 m to about 115 m, about 115 m to about 150 m, about 115 m to about 145 m, about 115 m to about 140 m, about 115 m to about 135 m, about 115 m to about 130 m, about 115 m to about 125 m, about 115 m to about 120 m, about 120 m to about 150 m, about 120 m to about 145 m, about 120 m to about 140 m, about 120 m to about 135 m, about 120 m to about 130 m, about 120 m to about 125 m, about 250 m to about 750 m, about 250 m to about 700 m, about 250 m to about 650 m, about 250 m to about 600 m, about 250 m to about 550 m, about 250 m to about 500 m, about 250 m to about 450 m, about 250 m to about 400 m, about 250 m to about 350 m, about 250 m to about 300 m, about 300 m to about 750 m, about 300 m to about 700 m, about 300 m to about 650 m, about 300 m to about 600 m, about 300 m to about 550 m, about 300 m to about 500 m, about 300 m to about 450 m, about 300 m to about 400 m, about 300 m to about 350 m, about 350 m to about 750 m, about 350 m to about 700 m, about 350 m to about 650 m, about 350 m to about 600 m, about 350 m to about 550 m, about 350 m to about 500 m, about 350 m to about 450 m, about 350 m to about 400 m, about 400 m to about 750 m, about 400 m to about 700 m, about 400 m to about 650 m, about 400 m to about 600 m, about 400 m to about 550 m, about 400 m to about 500 m, about 400 m to about 450 m, about 450 m to about 750 m, about 450 m to about 700 m, about 450 m to about 650 m, about 450 m to about 600 m, about 450 m to about 550 m, about 450 m to about 500 m, about 500 m to about 750 m, about 500 m to about 700 m, about 500 m to about 650 m, about 500 m to about 600 m, about 500 m to about 550 m, about 550 m to about 750 m, about 550 m to about 700 m, about 550 m to about 650 m, about 550 m to about 600 m, about 600 m to about 750 m, about 600 m to about 700 m, about 600 m to about 650 m, about 650 m to about 750 m, or about 650 m to about 700 m indicates that the treatment or treatment regimen is effective in treating the neurodegenerative disease in the subject. A subject having an increase in walking distance by at least about 1 m, at least about 2 m, at least about 3 m, at least about 4 m, at least about 5 m, at least about 10 m, at least about 15 m, at least about 20 m, at least about 25 m, at least about 30 m, at least about 35 m, at least about 40 m, at least about 45 m, at least about 50 m, at least about 55 m, at least about 60 m, at least about 65 m, at least about 70 m, at least about 75 m, at least about 80 m, at least about 85 m, at least about 90 m, at least about 95 m, at least about 100 m, at least about 105 m, at least about 110 m, at least about 115 m, at least about 120 m, at least about 125 m, at least about 130 m, at least about 135 m, at least about 140 m, at least about 145 m, at least about 150 m, at least about 155 m, at least about 160 m, at least about 165 m, at least about 170 m, at least about 175 m, at least about 180 m, at least about 185 m, at least about 190 m, at least about 195 m, or at least about 200 m indicates that the treatment or treatment regimen is effective in treating the neurodegenerative disease in the subject.

For example, a subject having an increase in walking speed (s/8 m) of at least about 30 s/8 m to at least about 10 s/8 m, at least about 25 s/8 m to at least about 10 s/8 m, at least about 20 s/8 m to at least about 10 s/8 m, at least about 15 s/8 m to at least about 10 s/8 m, at least about 30 s/8 m to at least about 15 s/8 m, at least about 25 s/8 m to at least about 15 s/8 m, at least about 20 s/8 m to at least about 15 s/8 m, at least about 30 s/8 m to at least about 20 s/8 m, at least about 25 s/8 m to at least about 20 s/8 m, or at least about 30 s/8 m to at least about 25 s/8 m indicates that the treatment or treatment regimen is effective in treating the neurodegenerative disease in the subject. A subject having an walking speed of less than about 30.0 s/8 m, less than about 25.0 s/8 m, less than about 20.0 s/8 m, less than about 15.0 s/8 m, less than about 14.5 s/8 m, less than about 14.0 s/8 m, less than about 13.5 s/8 m, less than about 13.0 s/8 m, less than about 12.9 s/8 m, less than about 12.8 s/8 m, less than about 12.7 s/8 m, less than about 12.6 s/8 m, less than about 12.5 s/8 m, less than about 12.4 s/8 m, less than about 12.3 s/8 m, less than about 12.2 s/8 m, less than about 12.1 s/8 m, less than about 12.0 s/8 m, less than about 11.9 s/8 m, less than about 11.8 s/8 m, less than about 11.7 s/8 m, less than about 11.6 s/8 m, less than about 11.5 s/8 m, less than about 11.4 s/8 m, less than about 11.3 s/8 m, less than about 11.2 s/8 m, less than about 11.1 s/8 m, less than about 11.0 s/8 m, less than about 10.5 s/8 m, less than about 10.0 s/8 m, less than about 9.5 s/8 m, less than about 9.0 s/8 m, less than about 8.5 s/8 m, or less than about 8.0 s/8 m indicates that the treatment or treatment regimen is effective in treating the neurodegenerative disease in the subject.

For example, a decrease in time it takes a subject to walk 8 m by at least about 0.5 sec, at least about 1 sec, at least about 2 sec, at least about 3 sec, at least about 4 sec, at least about 5 sec, at least about 6 sec, at least about 7 sec, at least about 8 sec, at least about 9 sec, at least about 10 sec, at least about 11 sec, at least about 12 sec, at least about 13 sec, at least about 14 sec, at least about 15 sec, at least about 16 sec, at least about 17 sec, at least about 18 sec, at least about 19 sec, at least about 20 sec, at least about 21 sec, at least about 22 sec, at least about 23 sec, at least about 24 sec, or at least about 25 sec indicates that the treatment or treatment regimen is effective in treating the neurodegenerative disease in the subject.

b. Controls/Reference Levels

It may be desirable to include a control sample. The control sample may be analyzed concurrently with the sample from the subject as described above. The results obtained from the subject sample can be compared to the results obtained from the control sample. Standard curves may be provided, with which assay results for the biological sample may be compared. Such standard curves present levels of marker as a function of assay units, i.e. chemiluminescent signal intensity, if a chemiluminescent label is used. Using samples taken from multiple donors, standard curves can be provided for control levels of the RGMa fragment in normal healthy tissue or MS tissue that has not been treated.

Thus, in view of the above, a method for determining the presence, amount, or concentration of RGMa fragment in a test sample is provided. The method comprises assaying (1) the test sample for RGMa fragment by Western blot analysis, for example, employing at least one capture antibody that binds to an epitope on RGMa fragment and at least one detection antibody that binds to the capture antibody or an epitope on the RGMa fragment, which is different from the epitope for the capture antibody, and optionally includes a detectable label. The method further comprises (2) comparing a signal generated by the detectable label as a direct or indirect indication of the presence, amount or concentration of RGMa fragment in the test sample to a signal generated as a direct or indirect indication of the presence, amount or concentration of RGMa fragment in a calibrator. The calibrator is optionally, and is preferably, part of a series of calibrators in which each of the calibrators differs from the other calibrators in the series by the concentration of RGMa fragment.

(1) Reference Levels

The methods described herein use reference levels of the RGMa fragment of a subject to (1) identify and determine the effectiveness of a treatment or treatment regimen for a subject suffering from a neurodegenerative disease; (2) predict the responsiveness of a subject suffering from a neurodegenerative disease to a treatment or treatment regimen; (3) provide a treatment or treatment regimen to a subject suffering from a neurodegenerative disease; (4) optimize a treatment or treatment regimen to a subject suffering from a neurodegenerative disease; (5) monitor a regeneration-promoting drug treatment or treatment regimen to a subject suffering from a neurodegenerative disease; and (6) screen a compound for therapeutic efficacy against a neurodegenerative disease.

Generally, predetermined or reference levels can be employed as a benchmark against which to assess results obtained upon assaying a test sample for the RGMa fragment (such as 18 kDa RGMa fragment, 30 kDa RGMa fragment, and/or 40 kDa RGMa fragment). Generally, in making such a comparison, the predetermined levels are obtained by running a particular assay a sufficient number of times and under appropriate conditions such that a linkage or association of the analyte present, amount or concentration with a particular stage or endpoint of MS with particular indicia can be made. Typically, the predetermined levels are obtained with assays of reference subjects (or populations of subjects). The reference subject may be a control subject, such as a normal or healthy subject who does not have a neurological disease or a subject having a neurological disease, such as a MS subject. The MS subject is a subject having a particular stage or pre-stage of MS (i.e., RRMS, SPMS, PPMS, PRMS, or CIS) that may or may not be treated for MS. The reference population or reference group may be a control group or a MS group. The MS group may include MS subjects having a particular stage or pre-stage of MS (i.e., RRMS, SPMS, PPMS, PRMS, or CIS), and/or subjects that have MS but are not treated for MS. The reference level may be the RGMa fragment levels in the subject before the treatment or treatment regimen is administered to the subject.

In particular, with respect to predetermined levels as employed for treatment responsiveness, the amount or concentration of the RGMa fragment, as discussed above, may be “unchanged,” “favorable” (or “favorably altered”), or “unfavorable” (or “unfavorably altered”). “Elevated” or “increased” refers to an amount or a concentration in a test sample that is higher or greater than a typical or normal level or range (e.g., predetermined level), such as a typical or normal level found in a control group or MS group, or is higher or greater than another reference level or range (e.g., earlier or baseline sample). “Elevated” or “increased” may also refer to an amount or a concentration in a test sample that is higher or greater than the level or range found in the subject before treatment has started. The term “lowered” or “reduced” refers to an amount or a concentration in a test sample that is lower or less than a typical or normal level or range (e.g., predetermined level), such as a typical or normal level found in a control group or MS group, or is lower or less than another reference level or range (e.g., earlier or baseline sample). The term “lowered” or “reduced” may also refer to an amount or a concentration in a test sample that is lower or less than the level or range found in the subject before treatment has started. The term “altered” refers to an amount or a concentration in a sample that is altered (increased or decreased) over a typical or normal level or range (e.g., predetermined level), such as a typical or normal level found in a control group or MS group, or over another reference level or range (e.g., earlier or baseline sample).

The typical or normal levels or ranges or another reference level or range (e.g., earlier or baseline sample) for the RGMa fragment, as discussed above, are defined in accordance with standard practice. A so-called altered level or alteration can be considered to have occurred when there is any net change as compared to the typical or normal level or range, or reference level or range that cannot be explained by experimental error or sample variation. In some embodiments, the level measured in a particular sample will be compared with the level or range of levels determined in similar samples from a so-called normal subject, i.e., control subject. In this context, a “normal” (sometimes termed “control” or “healthy”) subject is an individual with no detectable MS, and a “normal” patient or population is/are one(s) that exhibit(s) no detectable MS, for example. An “apparently normal subject” is one in which RGMa fragment has not been or is being assessed (such as 18 kDa RGMa fragment, 30 kDa RGMa fragment, and/or 40 kDa RGMa fragment). The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. In some embodiments, the level measured in a particular sample will be compared with the level or range of levels determined in similar samples from a MS subject or from earlier or baseline samples taken from the subject before the treatment has started.

In some embodiments, if the reference level is the RGMa fragment levels in a MS subject that is or is not treated for MS, levels higher than the reference levels of the RGMa fragment (such as 18 kDa RGMa fragment, 30 kDa RGMa fragment, and/or 40 kDa RGMa fragment) identify the subject as not being responsive to the treatment or treatment regimen or identify the treatment as not being effective for treating MS; levels lower than or equal to the reference level of the RGMa fragment (such as 18 kDa RGMa fragment, 30 kDa RGMa fragment, and/or 40 kDa RGMa fragment) identify the subject as being responsive to the treatment or treatment regimen or identify the treatment as being effective for treating MS.

In some embodiments, a change in the relative RGMa fragment level in the sample compared to a control, baseline, or earlier level or range identifies the subject as being responsive to the treatment or treatment regimen or identifies the treatment as being effective for treating MS. In some embodiments, a relative RGMa fragment level in the sample taken from the subject of less than about 100%, less than about 95%, less than about 85%, less than about 80%, less than about 75%, less than about 70%, less than about 65%, less than about 55%, less than about 50%, less than about 45%, less than about 40%, less than about 35%, less than about 30%, less than about 25%, less than about 20%, less than about 15%, less than about 10%, or less than about 5% as compared to the RGMa fragment levels in a control, earlier or baseline sample identify the subject as responsive or predicted to be responsive to the treatment or treatment regimen or identify the treatment as being effective for treating MS.

c. Sample

The method of using the RGMa fragment based diagnostic assay may include obtaining one or more samples from the subject. The one or more samples may be a cerebrospinal fluid (CSF) sample. In other embodiments, the one or more samples may be taken from any source, for example, tissue, blood, plasma, saliva, sputa, mucus, sweat, urine, urethral swabs, cervical swabs, urogenital or anal swabs, conjunctival swabs, ocular lens fluid, or cerebral spinal fluid. The one or more samples may be used (i) directly as obtained from the subject or (ii) following a pre-treatment to modify the character of the one or more samples. Thus, the one or more samples can be pre-treated by, for example, preparing plasma or serum from blood, disrupting cells, preparing liquids from solid materials, diluting viscous fluids, filtering liquids, adding reagents, purifying nucleic acids, purifying proteins, and so forth.

The samples may be taken at various time points before and after treatment or treatment regimen is administered to the subject. For example, the sample may be taken 1 day before, 0 day before, 1 day after, 2 days after, 3 days after, 4 days after, 5 days after, 6 days after, 7 days after, 8 days after, 9 days after, or 10 days after the treatment or treatment regimen has been administered to the subject.

d. Combination with Other Biomarkers

The methods described herein may also include using the RGMa fragment based diagnostic assay in combination with another biomarker to (1) identify and determine the effectiveness of a treatment or treatment regimen for a subject suffering from a neurodegenerative disease; (2) predict the responsiveness of a subject suffering from a neurodegenerative disease to a treatment or treatment regimen; (3) provide a treatment or treatment regimen to a subject suffering from a neurodegenerative disease; (4) optimize a treatment or treatment regimen to a subject suffering from a neurodegenerative disease; (5) monitor a regeneration-promoting drug treatment or treatment regimen to a subject suffering from a neurodegenerative disease; and (6) screen a compound for therapeutic efficacy against a neurodegenerative disease. In some embodiments, the biomarker may be a biomarker of MS, such as NOGO A, the ligand for the Nogo receptor (NgR), NOGO receptor, oligodendrocyte myelin glycoprotein (OMgp), myelin basic protein (MBP), Neurofilaments (Nf), growth-associated protein 43 (GAP-43); osteopontin; interleukin-17, Interleukin-6, Interferon-γ, and TNF-α. In some embodiments, a change, i.e., an increase or decrease, in the levels of the biomarker of MS in combination with the change in levels of the RGMa fragment(s) indicates whether the treatment or treatment regimen is effective.

e. Treatment Regimens

The method of using the RGMa fragment based diagnostic assay may be used in a treatment or treatment regimen for a neurodegenerative disease. The treatment or treatment regimen may include a neurorestorative drug, including a neuroregenerative drug, a neuroprotective drug, or combinations thereof. Neurorestoration encompasses correction of dysfunctional neuronal networks by changes in synaptic strengths, shifts in synaptic activity, activation of silent synapses, silencing of inhibitory synapases. Neurorestoration includes neuroregenerative processes. Neuroregeneration is the repair of damaged neuronal networks by regrowth of damaged fibers, by collateral sprouting of damaged fiber tracts or of healthy neighboring non-damaged tracts, formation of new synapses after regrowth and later formation of a new myelin sheets. Neuroprotection is the relative preservation of neuronal structure and/or function to prevent or slow disease progression and secondary injuries by halting or at least slowing the loss of neurons.

The treatment or treatment regimen may include, corticosteroids, such as triamcinolone acetonide (TCA); Tecfidera/BG-12 (dimethyl fumarate), Gilenya (fingolimod), Laquinimod, Daclizumab, Alemtuzumab, Rituximab, prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-23, IL-15, IL-16, IL-18, EMAP-11, GM-CSF, FGF, and PDGF, antibodies to cell surface molecules such as CD2, CD3, CD4, CDS, CD19, CD20, CD25, CD28, CD30, CD40, CD45, CD69, CD80, CD86, CD90 or their ligands, natalizumab, agents, such as methotrexate, cyclosporine, FK506, rapamycin, mycophenolate mofetil, teriflunomide, leflunomide, glatiramer acetate, Gilenya (fingolimod), mitoxantrone, peginterferon beta-1a, dimethyl fumarate, NSAIDs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, siL-1RI, siL-1RII, siL-6R), anti-inflammatory cytokines (e.g. IL-4, IL-10, IL-13 and TGFβ), or any combination thereof. The treatment or treatment regimen may include pro-regenerative RGMa antibodies. Pro-regenerative RGMa antibodies are described in U.S. Patent Publication Nos. 2004/0102376, 2010/0028340, 2011/0135664, 2013/0330347, and 2014/0023659.

The treatment may further include: therapeutic agent, imaging agent, cytotoxic agent, angiogenesis inhibitors; kinase inhibitors; co-stimulation molecule blockers; adhesion molecule blockers; anti-cytokine antibody or functional fragment thereof; methotrexate; cyclosporin; rapamycin; FK506; detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analogue, a cytokine, a cytokine antagonist, neuroprotective agent, such as an antioxidant, radical scavengers, an anticonvulsive drug like Phenytoin, anemia drug Erythropoetin, or combination thereof.

(1) TCA

The treatment or treatment regimen of a neurodegenerative disease may be triamcinolone acetonide treatments. The intrathecal application of a depot corticosteroid, called triamcinolone acetonide (TCA=VOLON A) may be used in MS patients. Numerous TCA studies revealed significant improvements in treated patients with decreased EDSS (Expanded Disability Status Score), increased walking distance and decreased walking speed. However, the underlying molecular mechanism for this improved functional recovery of the MS patients was completely unclear

Previous open observational trials described the benefits of repeated intrathecal application of the sustained release steroid triamcinolone acetonide (TCA) in primary and secondary progressive MS patients, particularly when they suffer from spinal symptoms. Clinically, there are three kinds of response to TCA therapy: (I) patients may report an immediate improvement during a series of four to six TCA applications or (II) a delayed amelioration of disease symptoms after several TCA injections or (III) no benefit. The biochemical and pharmacological reasons for these three behavioral response patterns to TCA therapy are not known in detail. Reduced CSF synthesis of free radicals was shown in patients with a distinct immediate enhancement of upper and lower limb function. Generally, free radicals have the capacity to mediate tissue destruction and to regulate production of various CSF proteins. Repeated intrathecal application of the sustained release steroid triamcinolone acetonide may be beneficial in progressive multiple sclerosis patients.

f. Methods of Determining Effectiveness of a Treatment for Neurodegenerative Disease

The method of using the RGMa fragment based diagnostic assay may be used in a method for determining effectiveness of the treatment or treatment regimen for the neurodegenerative disease in the subject in need thereof. The method of determining may include determining a level of at least one RGMa fragment in the sample obtained from the subject. Determining may include using the RGMa fragment based diagnostic assay to detect or measure the presence and/or level of the at least one RGMa fragment in the sample.

The method of determining may also include comparing the level of the at least one RGMa fragment to a control level of the at least one RGMa fragment. If the level of the at least one RGMa fragment is increased compared to the control level of the at least one RGMa fragment, then the treatment or treatment regimen may be determined to be ineffective in treating the neurodegenerative disease. When the treatment or treatment regimen is determined to be ineffective in treating the neurodegenerative disease, the method may also include administering a treatment or treatment regimen different than the ineffective treatment or treatment regimen to the subject.

If the level of the at least one RGMa fragment is decreased compared to the control level of the at least one RGMa fragment, then the treatment or treatment regimen may be determined to be effective in treating the neurodegenerative disease. When the treatment or treatment regimen is determined to be effective in treating the neurodegenerative disease, the method may also include continuing to administer the effective treatment or treatment regimen to the subject.

g. Method of Predicting Responsiveness of a Subject Suffering from a Neurodegenerative Disease to a Treatment

The method of using the RGMa fragment based diagnostic assay may be used in a method of predicting responsiveness of a subject suffering from the neurodegenerative disease to the treatment. The method may include determining the level of at least one RGMa fragment in the sample obtained from the subject. Determining may include using the RGMa fragment based diagnostic assay to detect or measure the presence and/or level of the at least one RGMa fragment in the sample.

The method of predicting may also include comparing the level of the at least one RGMa fragment to a control level of the at least one RGMa fragment. The method of predicting may further include providing a prediction of responsiveness of the subject to the treatment or treatment regimen if the level of the at least one RGMa fragment is decreased compared to the control level of the RGMa fragment. When the prediction of responsiveness is provided, the method of predicting may also include administering the treatment or treatment regimen to the subject.

h. Method of Treating a Subject Suffering from a Neurodegenerative Disease

The method of using the RGMa fragment based diagnostic assay may be used in a method of treating a subject suffering from the neurodegenerative disease. The method may include determining the level of at least one RGMa fragment in the sample obtained from the subject. Determining may include using the RGMa fragment based diagnostic assay to detect or measure the presence and/or level of the at least one RGMa fragment in the sample.

The method may also include comparing the level of the at least one RGMa fragment to a control level of the at least one RGMa fragment. The method may further include administering the treatment or treatment regimen to the subject if the level of the at least one RGMa fragment is increased compared to the control level of the RGMa fragment.

i. Method of Optimizing a Treatment Regimen for a Subject Suffering from a Neurodegenerative Disease

The method of using the RGMa fragment based diagnostic assay may be used in a method of optimizing the treatment or treatment regimen for the subject suffering from the neurodegenerative disease. The method may include determining a first level of at least one RGMa fragment in a first sample obtained from the subject. The first sample may be taken at a time point before or during a period when the subject has begun the treatment or treatment regimen. The method may also include determining a second level of the at least one RGMa fragment in a second sample obtained from the subject. The second sample may be obtained from the subject at a time point that is later than the first time point. The second sample may be taken at least about 1 hr, at least about 2 hr, at least about 3 hr, at least about 4 hr, at least about 5 hr, at least about 6 hr, at least about 7 hr, at least about 8 hr, at least about 9 hr, at least about 10 hr, at least about 12 hr, at least about 24 hr, at least about 2 days, at least about 3 days, at least about 4 days, at least about 5 days, at least about 4 days, at least about 5 days, at least about 2 weeks, at least about 1 month, or at least about a year from when the first sample was taken. Determining the first and second levels may include using the RGMa fragment based diagnostic assay to detect or measure the presence and/or level of the at least one RGMa fragment in the respective sample.

When the second level of the at least one RGMa fragment is less than the first level of the at least one RGMa fragment, then the treatment or treatment regimen may be effective against the neurodegenerative disease and the treatment regimen is not changed. When the second level of the at least one RGMa fragment is more than or equal to the first level of the at least one RGMa fragment, then the treatment or treatment regimen may not be effective against the neurodegenerative disease and the treatment regimen is changed.

j. Method of Monitoring a Regeneration-Promoting Drug Treatment of a Subject Suffering from a Neurodegenerative Disease

The method of using the RGMa fragment based diagnostic assay may be used in a method of monitoring the regeneration-promoting drug treatment of the subject suffering from the neurodegenerative disease. The method may include determining a first level of at least one RGMa fragment in a first sample obtained from the subject. The first sample may be taken at a time point before or during a period when the subject has begun the treatment or treatment regimen. The method may also include determining a second level of the at least one RGMa fragment in a second sample obtained from the subject. The second sample may be obtained from the subject at a time point that is later than the first time point. The second sample may be taken at least about 1 hr, at least about 2 hr, at least about 3 hr, at least about 4 hr, at least about 5 hr, at least about 6 hr, at least about 7 hr, at least about 8 hr, at least about 9 hr, at least about 10 hr, at least about 12 hr, at least about 24 hr, at least about 2 days, at least about 3 days, at least about 4 days, at least about 5 days, at least about 4 days, at least about 5 days, at least about 2 weeks, at least about 1 month, or at least about a year from when the first sample was taken. Determining the first and second levels may include using the RGMa fragment based diagnostic assay to detect or measure the presence and/or level of the at least one RGMa fragment in the respective sample.

A decrease in the second level of the at least one RGMa fragment compared to the first level of the at least one RGMa fragment may indicate that the treatment or treatment regimen does have therapeutic efficacy against the neurodegenerative disease. When the treatment or treatment regimen is determined to be therapeutically effective against the neurodegenerative disease, the method of monitoring may including continuing to administer the therapeutically effective treatment or treatment regimen to the subject.

An increase in the second level of the at least one RGMa fragment compared to the first level of the at least one RGMa fragment may indicate that the treatment or treatment regimen does not have therapeutic efficacy against the neurodegenerative disease. When the treatment or treatment regimen is determined to not be therapeutically effective against the neurodegenerative disease, the method of monitoring may include administering a treatment or treatment regimen to the subject that is different than the therapeutically ineffective treatment or treatment regimen.

k. Methods of Screening a Compound for Therapeutic Efficacy Against a Neurodegenerative Disease

The method of using the RGMA fragment based diagnostic assay may be used in a method of screening for the compound having therapeutic efficacy against the neurodegenerative disease. For example, the RGMa fragment based diagnostic assay may be used to evaluate neuroregenerative clinical drug candidates, neuroplasticity enhancing clinical drug candidates, or remyelination promoting clinical drug candidates. The method may include determining a first level of at least one RGMa fragment in a sample comprising cells. The method may also include contacting the sample with the compound. The method may further include determining a second level of the at least one RGMa fragment in the sample. The second level may be measured at least about 1 hr, at least about 2 hr, at least about 3 hr, at least about 4 hr, at least about 5 hr, at least about 6 hr, at least about 7 hr, at least about 8 hr, at least about 9 hr, at least about 10 hr, at least about 12 hr, at least about 24 hr, at least about 2 days, at least about 3 days, at least about 4 days, at least about 5 days, at least about 4 days, at least about 5 days, at least about 2 weeks, at least about 1 month, or at least about a year after the sample is contacted with the compound. Determining the first and second levels may include using the RGMa fragment based diagnostic assay to detect or measure the presence and/or level of the at least one RGMa fragment in the respective sample.

A decrease in the second level of the at least one RGMa fragment compared to the first level of the at least one RGMa fragment may indicate that the compound has therapeutic efficacy against the neurodegenerative disease. An increase in the second level of the at least one RGMa fragment compared to the first level of the at least on RGMa fragment may indicate that the compound does not have therapeutic efficacy against the neurodegenerative disease.

### 4. Kits for Performing the Methods

Provided herein is a kit, which may be used for performing the methods described above. The kit may provide (1) reagents capable of specifically binding to any of the RGMa fragments, such as each of 18 kDa RGMa fragment, 30 kDa RGMa fragment, and/or 40 kDa RGMa fragment to quantify the levels of the RGMa fragment, such as each of 18 kDa RGMa fragment, 30 kDa RGMa fragment, and/or 40 kDa RGMa fragment, in a biological sample isolated from a subject and (2) a reference standard indicating reference levels of the RGMa fragment, such as each of 18 kDa RGMa fragment, 30 kDa RGMa fragment, and/or 40 kDa RGMa fragment, wherein at least one reagent comprises at least one antibody capable of specifically binding the appropriate marker. The kit may comprise a reagent that is capable of specifically binding to at least one RGMa fragment, a reagent to quantify the concentration of each biomarker in the biological sample and a reference standard indicating the reference level of the RGMa fragment in the biological sample (i.e., 18 kDa RGMa fragment, 30 kDa RGMa fragment, and/or 40 kDa RGMa fragment). The kit may further comprise at least one reagent capable of specifically binding (i.e., an antibody) at least one additional biomarker of MS such as NOGO A, NOGO receptor, OMgp, MBP, Nf, GAP-43, osteopontin; interleukin-17, Interleukin-6, Interferon-γ, and TNF-α, and a reference standard indicating a reference level of the at least one additional biomarker of MS, if present.

The kit may comprise the antibodies and a means for administering the antibodies. The kit can further comprise instructions for using the kit and conducting the analysis, monitoring, or treatment.

The kit may also comprise one or more containers, such as vials or bottles, with each container containing a separate reagent. The kit may further comprise written instructions, which may describe how to perform or interpret an analysis, monitoring, treatment, or method described herein.

For example, the kit can comprise instructions for assaying the test sample for 18 kDa RGMa fragment, 30 kDa RGMa fragment, and/or 40 kDa RGMa fragment by Western blot analysis. The instructions can be in paper form or computer-readable form, such as a disk, CD, DVD, or the like. The antibody can be an 18 kDa RGMa fragment, 30 kDa RGMa fragment, and/or 40 kDa RGMa fragment capture antibody and/or 18 kDa RGMa fragment, 30 kDa RGMa fragment, and/or 40 kDa RGMa fragment detection antibody (meaning an antibody labeled with a detectable label). For example, the kit can contain at least one capture antibody that specifically binds at least one RGMa fragment. The kit can also contain a conjugate antibody (such as an antibody labeled with a detectable label) for the capture antibody (namely, a conjugate antibody for the capture antibodies that specifically bind to 18 kDa RGMa fragment, 30 kDa RGMa fragment, and/or 40 kDa RGMa fragment.

Alternatively or additionally, the kit can comprise a calibrator or control, e.g., purified, and optionally lyophilized, (e.g., 18 kDa RGMa fragment, 30 kDa RGMa fragment, and/or 40 kDa RGMa fragment), and/or at least one container (e.g., tube, microtiter plates or strips, which can be already coated with an anti-18 kDa RGMa fragment, 30 kDa RGMa fragment, and/or 40 kDa RGMa fragment monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying 18 kDa RGMa fragment, 30 kDa RGMa fragment, and/or 40 kDa RGMa fragment.

As alluded to above, any antibodies, which are provided in the kit, such as recombinant antibodies specific for 18 kDa RGMa fragment, 30 kDa RGMa fragment, and/or 40 kDa RGMa fragment can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the Western blot kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a blood sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle, chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of MS, such as NOGO A, NOGO receptor, OMgp, MBP, Nf, GAP-43, osteopontin; interleukin-17, Interleukin-6, Interferon-γ, and TNF-α. Examples of analytes include, but are not limited to 18 kDa RGMa fragment, 30 kDa RGMa fragment, and/or 40 kDa RGMa fragment as well other analytes and biomarkers discussed herein, or otherwise known in the art. In some embodiments one or more components for assaying a test sample for 18 kDa RGMa fragment, 30 kDa RGMa fragment, and/or 40 kDa RGMa fragments enable the determination of the presence, amount or concentration of 18 kDa RGMa fragment, 30 kDa RGMa fragment, and/or 40 kDa RGMa fragment. A sample, such as a serum sample, can also be assayed for 18 kDa RGMa fragment, 30 kDa RGMa fragment, and/or 40 kDa RGMa fragment using TOF-MS and an internal standard.

It will be readily apparent to those skilled in the art that other suitable modification and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples which are merely intended only to illustrate some aspects and embodiments of the disclosure, and should not be viewed as limiting to the scope of the disclosure. The disclosures of all journal references, U.S. patents and publications referred to herein are hereby incorporated by reference in their entireties.

The present invention has multiple aspects, illustrated by the following non-limiting examples.

### EXAMPLES

### Example 1

### Materials and Methods

Subjects.

25 MS patients (age: 50±1.64 [mean±SEM, years] were studied. The average MS duration was 14.02±1.71 [years]. The subjects included 14 women and 11 men. The subjects included 13 secondary progressive [8 women, 5 men] and 12 primary progressive [6 women, 6 men]). Exclusion criteria were an acute onset of exacerbation or a recent clearly increased progression of their symptoms.

Design.

TCA application was followed by a mandatory stay in bed for at least six hours in order to support and ease the diffusion of TCA in the CSF and the spinal cord. Lumbar puncture was performed with an atraumatic Sprotte needle. The preexisting immune system modulating drug therapy remained stable. Spasticity reducing therapy was not changed. Expanded Disability Status Score (EDSS) ratings, maximum walking distance, and walking speed assessments were performed at baseline and on each day after a 40 mg Triamcinolone acetonide (TCA) administration, dissolved in 10 ml saline, up to the fourth TCA application.

CSF Sampling and CSF Analysis.

CSF was taken before the intrathecal TCA application. Aliquots of approximate 1 ml CSF were collected in sterile Eppendorf tubes, frozen immediately and stored at −20° C. RGMa determination was performed at baseline before the first TCA application (moment I) and on each day after a TCA injection (moments II, III, IV, and V). The protein concentration was determined by measurements with a NanoDrop Analyser (Thermo Scientific).

Western Blot Analysis.

CSF RGMa levels were analysed by Western blotting and immunodetection (Schaffar et al., J Neurochem 107:418-431 (2008)). Briefly, 10 μl of each CSF sample was mixed with 10 μl SDS-loading dye (Life Technologies) and incubated at 95° C. for 10 min. 10 μl of these samples were separated on SDS-PA-Gels (Life Technologies) and transferred to nitrocellulose membrane. After immune staining with anti-RGMa antibody (R&D Systems, BAF2459) and secondary reagent Ultra-Sensitive ABC Peroxidase staining Kit (Pierce, 32050) the membranes were incubated with a luminescence reagent (Thermo Scientific, SuperSignal West Femto Chemiluminescence Substrate, 34094).

Band Intensity Analysis.

The band intensities were measured with Quantity One Version 4.6.9 (BioRad). Briefly, “Frame lanes . . . ” in Band Analysis Quick Guide was selected and number of lanes was chosen. Lanes were adjusted finely with “Add/Adjust Anchors”-tool and bands of interest manually detected with “Detect Bands . . . ”-tool. With “All Lane Report” the Trace intensity x mm was detected and used for the analysis. For each patient, moment I was calculated as 100% and moments II, III and IV were related in percent to moment I. The average of these percentages was plotted into a graph and the statistical analysis was done with ANOVA with Bonferroni's multiple comparison test in GraphPad Prism 5.

Procedure for the statistical evaluation of laboratory outcomes. The CSF of all 25 patients was analysed by Western blotting for RGMa expression independently of the clinical scores. Equal amounts of CSF were loaded for the analysis by Western blotting to compare the RGMa levels in the same volume. The 30 kDa and 40 kDa bands in CSF of all 25 patients in this group were analyzed by densitometer measurements. The clinical data were grouped into immediate responders and not immediate non-responders during the observational interval.

Statistics.

ANCOVA with repeated measures design were employed for this exploratory analysis of this pilot trial. Covariates were MS duration, MS types, sex and age as covariates. The post hoc analysis was done with the Tukey's multiple comparisons test against baseline. The statistical analysis was performed with GraphPad Prism 5 Software.

### Example 2

### RGMa in Cerebrospinal Fluid in MS Patients

To determine if RGMa fragments exist in cerebrospinal fluid of patients suffering from MS, gel electrophoresis of human CSF samples, western blots and immunodetection with RGMa-specific antibodies were performed, as described in Example 2. FIG. 1 shows the presence of all RGMa fragments described above in human CSF (modified from Key and Lah, Cell Adhesion & Migration 6:2, 85-90 (2012)) Immunodetection with RGMa-specific antibodies resulted in detection of three fragments with sizes of approximately 40, 30 and 18 kDa. TCA=Triamcinolone acetonide, a depot corticosteroid used for intrathecal treatment of progressive MS patients, I-II, III, IV, V before first, second, third, fourth and fifth TCA treatment, respectively.

### Example 3

### Effects of Triamcinolone Acetonide in Multiple Sclerosis Patients

Many current drugs slow disease progression in MS but do not result in enhanced functional recovery in MS patients. Experiments were performed to demonstrate the efficacy of four triamcinolone applications every other day in association with RGMa levels in cerebrospinal fluid. Clinical evaluation was performed at baseline and on each day after a triamcinolone administration in 25 progressive multiple sclerosis patients. Before the TCA was administered to a patient, 1-2 ml aliquots of CSF were withdrawn. RGMa concentrations were determined before each triamcinolone application by western blot analysis with quantification. Dependent on the disease activity, the patients usually received 4-6 TCA applications.

Clinical Data of the Responders.

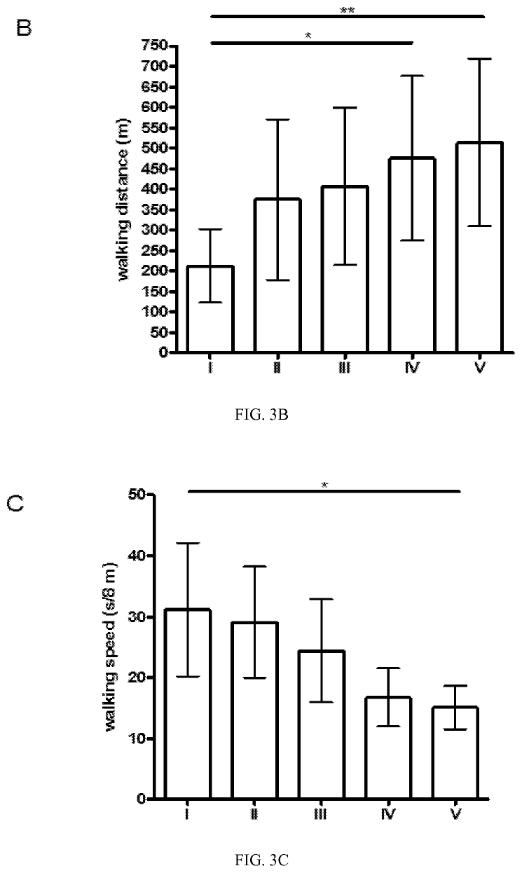

17 patients (10 men, 7 women; age: 52.18±2.04, MS duration: 15.74±1.92) improved following treatment. The EDSS scores (F=8.55; p<0.009 [FIG. 3A]) of these patients went down, the maximum walking distances increased (F=3.64; p=0.01 [FIG. 3B]), and the walking speed went up (F=3.42; p<0.01 [FIG. 3C]). Three patients were wheel chair bound and their data were not included in the analysis of walking abilities.

Clinical Data of the not Immediately Responding Patients.

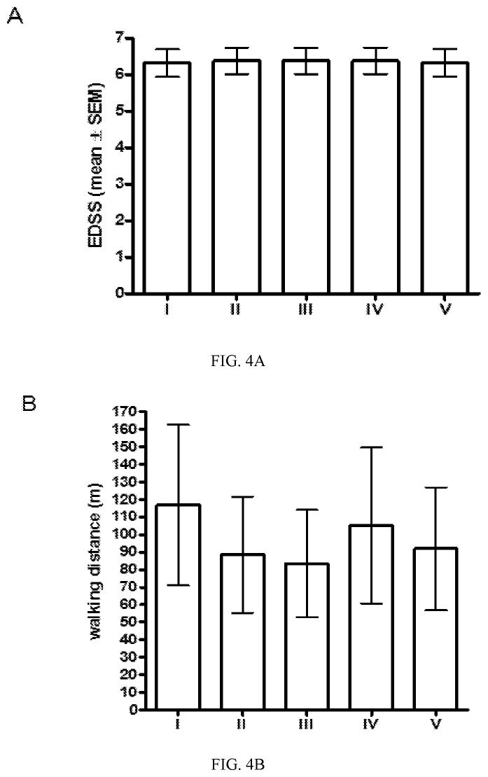

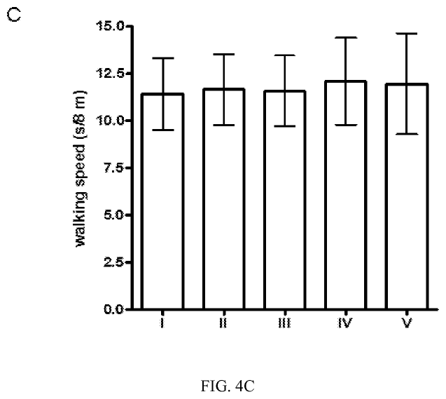

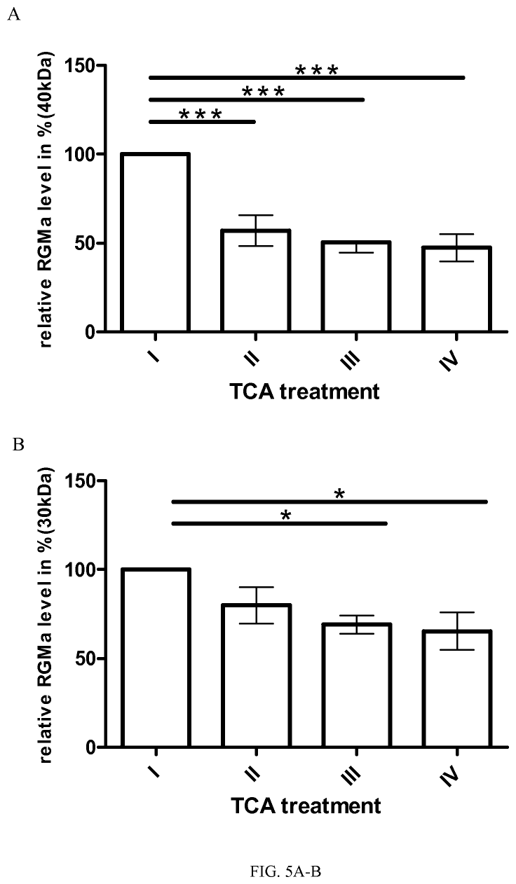

8 patients (1 man, 7 women; age: 45.38±2.04; MS duration: 10.38±3.24) did not immediately respond during the observation period. There was no significant change of EDSS scores (F=1; ns [FIG. 4A]); maximum walking distances (F=1.52; ns [FIG. 4B]) and walking speed (F=0.021; ns [FIG. 4C]). 6 patients reported a delayed improvement within three weeks after the TCA applications.

Generally, no serious side effects appeared in all participants. No impact of the covariates was found in the whole analysis.

Laboratory Outcomes.

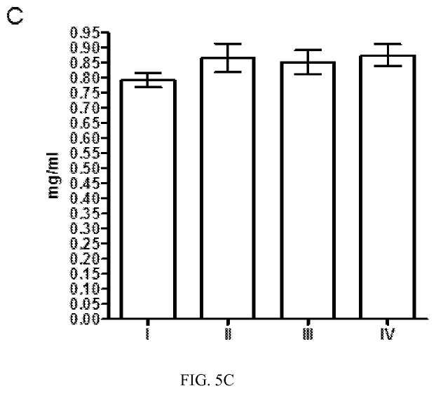

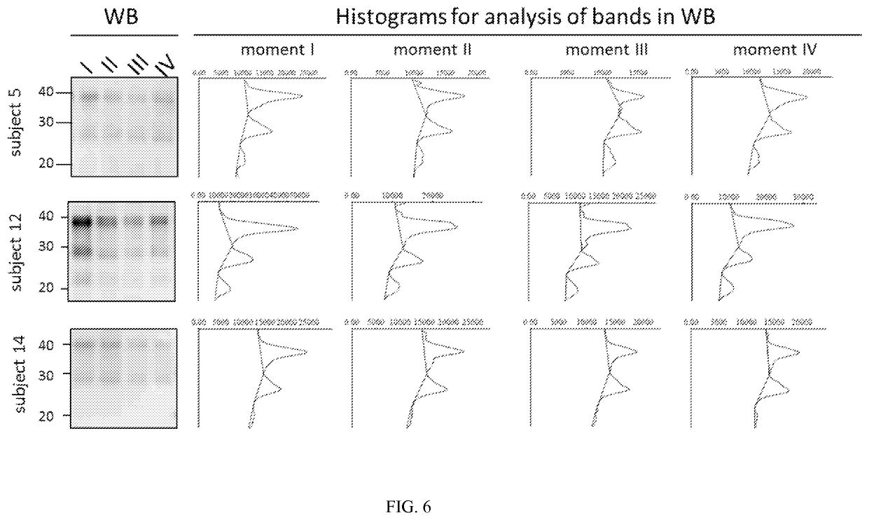

Responders. There was a reduction of RGMa levels in this cohort. The decline was less pronounced with the 30 kDa-(F=3.82; p<0.05 [FIG. 5B]) than with the 40 kDa form (F=9.12; p<0.0001 [FIG. 5A]). There was no significant change of the protein CSF concentrations (F=2.77; ns [FIG. 5C]). FIG. 6 illustrates three representative Western blots.

Not Immediately Responding Patients.

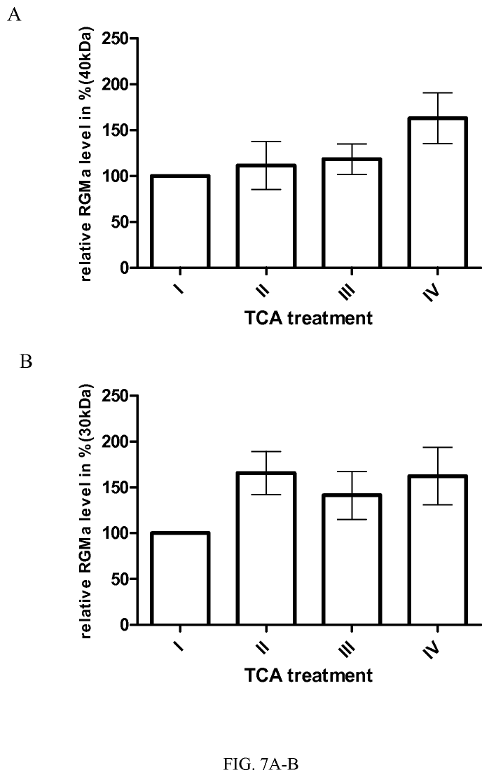

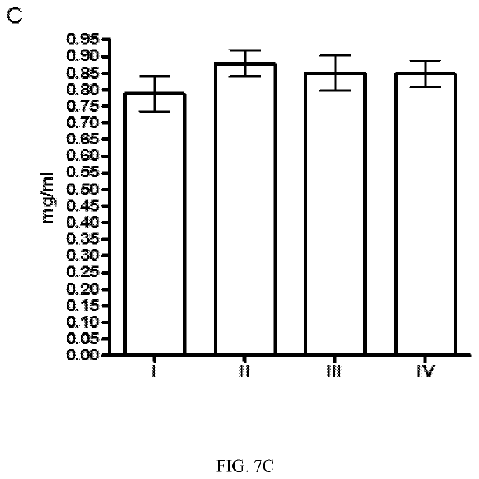

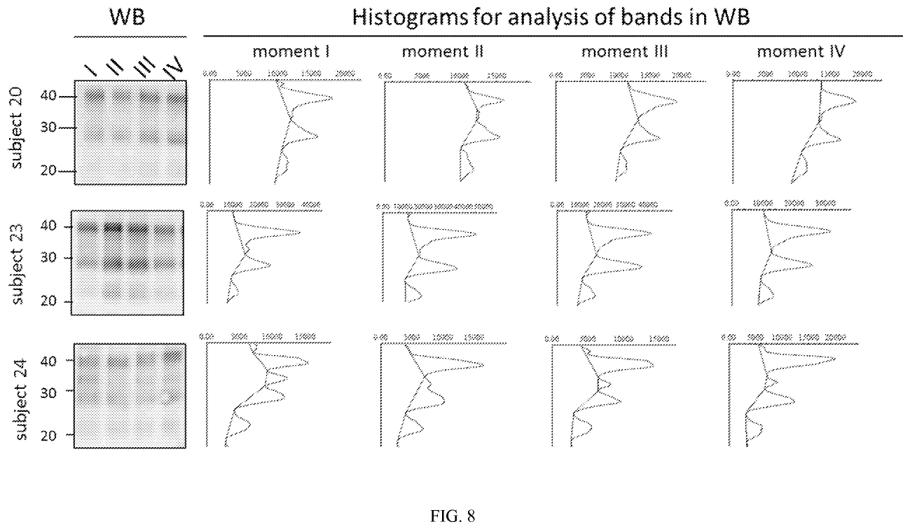

No significant changes appeared in both forms of RGMa (30 kDa: F=2.98; ns [FIG. 7B]; 40 kDa: F=0.84; ns [FIG. 7A] and in the protein content (F=2.86; ns [FIG. 7C] in the CSF. FIG. 8 shows three representative Western blots in this group.

Clinical scores for multiple sclerosis improved, and the maximum walking distance and -speed ameliorated in 17 patients. RGMa levels declined in these responders. The remaining patients showed no prompt clinical benefit and no decrease of RGMa concentrations. Decline of RGMa may reflect regeneration and functional recovery by triamcinolone in progressive multiple sclerosis patients. The protein concentration did not differ between responders and non-responders. There were no relevant alterations of cell counts in both cohorts respectively between both of them in the CSF.

Recurrent of TCA applications induced a decreased concentration of all the investigated RGMa fragments. It was surprising that a TCA-induced reduction of the concentration of soluble RGMa fragments in CSF was observed in those patients showing functional improvements, indicating that RGMa fragments may be used to evaluate the outcome of MS patients. This was further strengthened by the second observation, in which another group of MS patients also treated with TCA showed no decrease in RGMa fragment CSF concentration and concomitantly no functional recovery.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A method of detecting and quantifying at least one RGMa fragment in a sample, the method comprising: (a) obtaining a sample from a subject comprising at least one RGMa fragment; (b) contacting the sample with a capture binding protein, wherein the capture binding protein binds to the at least one RGMa fragment to form a capture binding protein-RGMa fragment complex; (c) contacting the sample with a detection binding protein, wherein the detection binding protein interacts with the capture binding protein to form a detection binding protein-capture binding protein RGMa fragment complex, and (d) detecting and quantifying the at least one RGMa fragment in the sample.

Clause 2. The method of clause 1, wherein the at least one RGMa fragment is a RGMa fragment having a size between about 1 kDa to about 65 kDa.

Clause 3. The method of clause 1 or 2, wherein the RGMa fragment has a size of 10 kDa, 18 kDa, 20 kDa, 30 kDa, 40 kDa, 50 kDa, or 65 kDa.

Clause 4. The method of any one of clauses 1 to 3, wherein the RGMA fragment is selected from the group consisting of 18 kDa RGMa fragment, 30 kDa RGMa fragment, and 40 kDa RGMa fragment.

Clause 5. The method of any one of clauses 1 to 4, wherein the at least one RGMa fragment is separated using gel electrophoresis before step (b).

Clause 6. The method of clause 5, further comprising immobilizing the at least one RGMa fragment to a membrane to generate a western blotting membrane before step (b); contacting the western blotting membrane with the capture binding protein, wherein the capture binding protein binds to the at least one RGMa fragment immobilized on the western blotting membrane to form a capture binding protein-RGMa fragment complex in step (b); and contacting the western blotting membrane with a detection binding protein, wherein the detection binding protein interacts with the capture binding protein to form a detection binding protein-capture binding protein RGMa fragment complex in step (c).

Clause 7. The method of any one of clauses 1 to 6, wherein at least two RGMa fragments are detected.

Clause 8. The method of clause 7, wherein the at least two RGMa fragments are 30 kDa and 40 kDa in size.

Clause 9. The method of any one of clauses 1 to 6, wherein at least three RGMa fragments are detected.

Clause 10. The method of clause 9, wherein the at least three RGMa fragments are 18 kDa, 30 kDa, and 40 kDa in size.

Clause 11. The method of any one of clauses 1 to 10, wherein the at least one RGMa fragment is a soluble RGMa fragment.

Clause 12. The method of any one of clauses 5 to 11, further comprising separating a RGMa protein standard on the gel concurrently with the proteins in the sample in step (b); and (g) comparing the at least one RGMa fragment with the separated RGMa protein standard to quantify the fragments.

Clause 13. The method of clause 12, wherein the RGMa protein standard is a gradient of recombinant RGMa fragments.

Clause 14. The method of clause 13, wherein the gradient comprises the RGMa protein standard 10, 25, 50, 100, and 200 pg/mL.

Clause 15. The method of any one of clauses 1 to 14, wherein the size of the RGMa fragment is determined by SDS-PAGE.

Clause 16. The method of clause 15, wherein the SDS PAGE is 4-15%.

Clause 17. The method of any one of clauses 6 to 16, wherein the membrane is a nitrocellulose membrane.

Clause 18. The method of any one of clauses 1 to 17, wherein the capture binding protein is an RGMa-selective antibody.

Clause 19. The method of clause 18, wherein the antibody is a biotinylated RGMa-selective antibody.

Clause 20. The method of clause 19, wherein the detection binding protein is a tetravalent avidin and the detectable label is a biotinylated horseradish peroxidase.

Clause 21. The method of clause 20, wherein the at least one RGMa fragment is detected using a peroxidase staining kit.

Clause 22. A method of determining the effectiveness of a treatment for a neurodegenerative disease in a subject in need thereof, the method comprising: (a) determining the level of at least one RGMa fragment in a sample from the subject using the method of any one of clauses 1 to 21; and (b) comparing the level of the at least one RGMa fragment in a sample from the subject to a control level of the at least one RGMa fragment, wherein if the level of the at least one fragment is increased compared to the control level, the treatment is determined to be ineffective in treating the neurodegenerative disease, and wherein if the level of the at least one fragment is the same or decreased compared to the control level, the treatment is determined to be effective in treating the neurodegenerative disease.

Clause 23. The method of clause 22, further comprising continuing to administer the treatment determined to be effective in treating the neurodegenerative disease to the subject in need thereof.

Clause 24. The method of clause 22 or 23, wherein the control level of the at least one RGMa fragment is the level of the at least one RGMa fragment in a subject that has the neurodegenerative disease but has not been treated with for the neurodegenerative disease.

Clause 25. A method of predicting the responsiveness of a subject suffering from a neurodegenerative disease to a treatment; the method comprising: (a) determining the levels of at least one RGMa fragment in a sample from the subject using the method of any one of clauses 1 to 21; (b) comparing the levels of the at least one RGMa fragment in a sample from the subject to a control level of the at least one RGMa fragment; and (c) providing a prediction of responsiveness of the subject to a treatment if the levels of the at least one RGMa fragment in a sample are decreased compared to the control levels.

Clause 26. The method of clause 25, further comprising administering the treatment to the subject predicted to be responsive to the treatment.

Clause 27. A method of treating a subject suffering from neurodegenerative disease, the method comprising: (a) determining the levels of at least one RGMa fragment in a sample from the subject using the method of any one of clauses 1 to 21, (b) comparing the levels of the at least one RGMa fragment in a sample from the subject to a control level of the at least one RGMa fragment; and (c) administering a treatment regimen to the subject if the levels of the fragments are increased compared to control levels.

Clause 28. The method of any one of clauses 22 to 27, wherein the treatment comprises a, neurorestorative drug, neuroprotective drug, or neuroregenerative drug.

Clause 29. The method of any one of clauses 22 to 28, wherein the treatment comprises at least one of triamcinolone acetonide (TCA), Tecfidera/BG-12 (dimethyl fumarate), Gilenya (fingolimod), Laquinimod, β-Interferons, Copaxone, Daclizumab, Alemtuzumab, Rituximab, or combinations thereof.

Clause 30. The method of any one of clauses 26 to 29, wherein the treatment comprises triamcinolone acetonide (TCA).

Clause 31. A method of optimizing a treatment regimen for a subject suffering from a neurodegenerative disease, the method comprising: (a) determining a first level of at least one RGMa fragment in a first sample from the subject using the method of any one of clauses 1 to 20, wherein the first sample is taken from the subject at a time point before or during the period when the subject has begun a treatment regimen; (b) determining a second level of the at least one RGMa fragment in second sample from the subject at a time later than step (a), wherein an decrease in the second level of the at least one RGMa fragment compared to the first level of the at least one RGMa fragment indicates the treatment regimen has a therapeutic efficacy against the neurodegenerative disease; (c) determining the levels of at least one RGMa fragment in a first sample from the subject using the method of clause 1, (d) comparing the levels of the at least one RGMa fragment in a sample from the subject to a control level of the at least one RGMa fragment; and (e) providing a prediction of responsiveness of the subject to a treatment if the levels of the at least one RGMa fragment in a sample are decreased compared to the control levels.

Clause 32. The method of clause 31, wherein the treatment regimen is a neurorestorative treatment regimen.

Clause 33. The method of clause 32, wherein the success rate of the neurorestorative treatment regimen is increased.

Clause 34. The method of clause 31, wherein the treatment regimen is a neuroprotective treatment regimen.

Clause 35. The method of clause 34, wherein the success rate of the neuroprotective treatment regimen is increased.

Clause 36. A method of monitoring a regeneration-promoting drug treatment of a subject suffering from neurodegenerative disease, the method comprising: (a) determining a first level of at least one RGMa fragment in a first sample from the subject using the method of any one of clauses 1 to 21, wherein the first sample is taken from the subject at a time point before or during the period when the subject has begun drug treatment; (b) determining a second level of the at least one RGMa fragment in second sample from the subject at a time later than step (a), wherein an decrease in the second level of the at least one RGMa fragment compared to the first level of the at least one RGMa fragment indicates the drug treatment regimen has a therapeutic efficacy against the neurodegenerative disease, and an increase in the second level of the at least one RGMa fragment compared to the first level of the at least one RGMa fragment indicates the drug treatment regimen does not have a therapeutic efficacy against the neurodegenerative disease; and (c) administering a different drug treatment to the subject if the drug treatment regimen does not have a therapeutic efficacy against the neurodegenerative disease.

Clause 37. A method of screening a compound for therapeutic efficacy against a neurodegenerative disease, the method comprising: (a) determining a first level of at least one RGMa fragment in a sample comprising cells using the method of any one of clauses 1 to 21; (b) contacting the sample with a compound, (c) determining a second level of at least one RGMa fragment in second sample from the subject at a time later than step (b), wherein an decrease in the second level of the at least one RGMa fragment compared to the first level of the at least one RGMa fragment indicates the compound as having therapeutic efficacy against the neurodegenerative disease, and wherein an increase in the second level of the at least one RGMa fragment compared to the first level of the at least one RGMa fragment indicates the compound as not having therapeutic efficacy against the neurodegenerative disease; and (d) selecting the compound identified as having therapeutic efficacy.

Clause 38. The method of any one of clauses 22 to 37, wherein at least two RGMa fragment are detected.

Clause 39. The method of clause 38, wherein the at least two RGMa fragments are 30 kDa and 40 kDa in size.

Clause 40. The method of any one of clauses 22 to 37, wherein at least three RGMa fragments are detected.

Clause 41. The method of clause 40, wherein the at least three RGMa fragments are 18 kDa, 30 kDa, and 40 kDa in size.

Clause 42. The method of any one of clauses 22 to 41, wherein neurodegenerative disease or disorder is multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy.

Clause 43. The method of any one of clauses 22 to 42, wherein neurodegenerative disease or disorder is multiple sclerosis.

Clause 44. The method of any one of clauses 1 to 43, wherein the RGMa fragment is a human RGMa fragment.

Clause 45. The method of any one of clauses 1 to 44, wherein the sample comprises cerebrospinal fluid, blood, serum or plasma.

1.-26. (canceled)

27. A method of treating a subject suffering from neurodegenerative disease, the method comprising:
(a) determining the levels of at least one RGMa fragment in a sample from the subject using the method comprising
(a₁) obtaining a sample from a subject comprising at least one RGMa fragment;
(a₂) contacting the sample with a capture binding protein, wherein the capture binding protein binds to the at least one RGMa fragment to form a capture binding protein-RGMa fragment complex;
(a₃) contacting the sample with a detection binding protein, wherein the detection binding protein interacts with the capture binding protein to form a detection binding protein-capture binding protein RGMa fragment complex, and
(a₄) detecting and quantifying the at least one RGMa fragment in the sample,
(b) comparing the levels of the at least one RGMa fragment in a sample from the subject to a control level of the at least one RGMa fragment; and
(c) administering a treatment regimen to the subject if the levels of the fragments are increased compared to control levels.

28. The method of claim 27, wherein the treatment comprises a, neurorestorative drug, neuroprotective drug, or neuroregenerative drug.

29. The method of claim 27, wherein the treatment comprises at least one of triamcinolone acetonide (TCA), Tecfidera/BG-12 (dimethyl fumarate), Gilenya (fingolimod), Laquinimod, β-Interferons, Copaxone, Daclizumab, Alemtuzumab, Rituximab, or combinations thereof.

30. The method of claim 29, wherein the treatment comprises triamcinolone acetonide (TCA).

31.-37. (canceled)

38. The method of claim 27, wherein at least two RGMa fragment are detected.

39. The method of claim 38, wherein the at least two RGMa fragments are 30 kDa and 40 kDa in size.

40. The method of claim 27, wherein at least three RGMa fragments are detected.

41. The method of claim 40, wherein the at least three RGMa fragments are 18 kDa, 30 kDa, and 40 kDa in size.

42. The method of claim 27, wherein neurodegenerative disease or disorder is multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, spinal cord injury, traumatic brain injury, stroke, glaucoma, diabetic retinopathy, age-dependent macular degeneration, or a leukodystrophy.

43. The method of claim 42, wherein neurodegenerative disease or disorder is multiple sclerosis.

44. The method of claim 27, wherein the RGMa fragment is a human RGMa fragment.

45. The method of claim 27, wherein the sample comprises cerebrospinal fluid, blood, serum or plasma.

46. The method of claim 27, wherein the at least one RGMa fragment is a RGMa fragment having a size between about 1 kDa to about 65 kDa.

47. The method of claim 27, wherein the RGMa fragment has a size of 10 kDa, 18 kDa, 20 kDa, 30 kDa, 40 kDa, 50 kDa, or 65 kDa.

48. The method of claim 27, wherein the RGMA fragment is selected from the group consisting of 18 kDa RGMa fragment, 30 kDa RGMa fragment, and 40 kDa RGMa fragment.

49. The method of claim 27, wherein the at least one RGMa fragment is a soluble RGMa fragment

50. The method of claim 48, wherein the size of the RGMa fragment is determined by SDS-PAGE.

51. The method of claim 27, wherein the capture binding protein is an RGMa-selective antibody.

52. The method of claim 51, wherein the antibody is a biotinylated RGMa-selective antibody.

53. The method of claim 27, wherein the detection binding protein is a tetravalent avidin and the detectable label is a biotinylated horseradish peroxidase.
